(19) United States
(12) Patent Application Publication
(10) Pub. No.: US 2021/0402398 A1
(43) Pub. Date: Dec. 30, 2021

(54) DIAGNOSTIC DEVICE WITH DUAL-REGION SUBSTRATE

(71) Applicant: Detect, Inc., Guilford, CT (US)

(72) Inventors: Jonathan M. Rothberg, Guilford, CT (US); Isaac Bean, Colorado Springs, CO (US)

(73) Assignee: Detact, Inc., Guilford, CT (US)

(21) Appl. No.: 17/357,203

(22) Filed: Jun. 24, 2021

Related U.S. Application Data
(60) Provisional application No. 63/043,758, filed on Jun. 24, 2020.

(51) Int. Cl. B01L 3/00; C12Q 1/70

(52) U.S. Cl. CPC B01L 3/502715; C12Q 1/701; B01L 3/5025; B01L 2200/14; B01L 2300/0825; B01L 2200/085; B01L 3/527

(57) ABSTRACT

Described herein in an embodiment is a diagnostic device for detecting the presence of one or more target nucleic acids (e.g., a nucleic acid of a pathogen, such as SARS-CoV-2 or an influenza virus). In some cases, the diagnostic device comprises a substrate comprising both a reagent delivery region comprising one or more reagents (e.g., one or more nucleic acid amplification reagents) and a lateral flow assay region configured to detect one or more target nucleic acids. The substrate may be removably or permanently coupled to an inner component that is movable relative to an outer component. The diagnostic device may further comprise a sample-collecting component coupled to the outer component and/or the inner component. In some embodiments, the inner component may be moved relative to the outer component in order to sequentially expose the substrate to the collected sample. In some cases, the diagnostic device may be used with a reaction tube comprising one or more liquids (e.g., a reaction buffer) and/or a heating unit.

### RELATED APPLICATION

This application claims priority under 35 U.S.C. § 119(e) to U.S. Provisional Patent Application No. 63/043,758, filed Jun. 24, 2020, and entitled “Diagnostic Device with Dual-Region Substrate,” which is hereby incorporated by reference in its entirety.

### FIELD

The present invention generally relates to diagnostic devices for detecting the presence of a target nucleic acid.

### REFERENCE TO SEQUENCE LISTING SUBMITTED AS A TEXT FILE VIA EFS-WEB

The instant application contains a sequence listing which has been submitted in ASCII format via EFS-Web and is hereby incorporated by reference in its entirety. Said ASCII copy, created on Jun. 24, 2021, is named H096670037US01-SEQ-MKN and is 4,830 bytes in size.

### BACKGROUND

The ability to rapidly diagnose diseases-particularly highly infectious diseases—is critical to preserving human health. As one example, the high level of contagiousness, the high mortality rate, and the lack of a treatment for the novel coronavirus 2019 (COVID-19) have resulted in a pandemic that has already killed millions of people. The existence of rapid, accurate COVID-19 diagnostic tests could allow infected individuals to be quickly identified and isolated, which could assist with containment of the disease.

### SUMMARY

Diagnostic devices for detecting the presence of a target nucleic acid, and associated systems and methods, are generally described.

In some aspects, a diagnostic pen for detecting a first target nucleic acid is provided. In some embodiments, the diagnostic pen comprises an outer casing. In some embodiments, the diagnostic pen comprises an inner member movable within the outer casing. In some embodiments, the diagnostic pen comprises a sample-collecting component attached to the outer casing and/or the inner member.

In some aspects, a substrate is provided. In some embodiments, the substrate comprises a reagent delivery region comprising one or more reagents. In some embodiments, the substrate comprises a lateral flow assay region. In some embodiments, the substrate comprises a separation region positioned between the reagent delivery region and the lateral flow assay region.

In some aspects, a diagnostic device is provided. In some embodiments, the diagnostic device comprises an outer component. In some embodiments, the diagnostic device comprises an inner component comprising a substrate. In certain cases, the substrate comprises a reagent delivery region comprising one or more reagents. In certain cases, the substrate comprises a lateral flow assay region. In certain cases, the substrate further comprises a separation region between the reagent delivery region and the lateral flow assay region. In some embodiments, the inner component is movable relative to the outer component.

In some aspects, a diagnostic kit is provided. In some embodiments, the diagnostic kit comprises a diagnostic device. In certain cases, the diagnostic device comprises an outer component. In certain cases, the diagnostic device comprises an inner component. In some instances, at least a portion of the inner component is configured to detect a first target nucleic acid. In certain cases, the diagnostic device comprises a sample-collecting component attached to the outer component and/or the inner component. In certain cases, the inner component is movable relative to the outer component. In some embodiments, the diagnostic kit comprises a reaction tube comprising one or more liquids. In some embodiments, the diagnostic kit comprises a heating unit.

In some aspects, a method of testing is provided. In some embodiments, the method comprises collecting a sample with a sample-collecting component. In certain cases, the sample-collecting component is attached to an outer component and/or an inner component of a diagnostic device. In some embodiments, the method comprises moving the inner component relative to the outer component in a first movement such that at least a first portion of the inner component is exposed to fluidic contents of a reaction tube. In some embodiments, the method comprises reading an indication of the presence or absence of a first target nucleic acid in the sample.

In some aspects, a method of forming a diagnostic device is provided. In some embodiments, the method comprises providing an outer component. In some embodiments, the method comprises forming an inner component comprising a portion configured to detect a first target nucleic acid. In some embodiments, the method comprises inserting the inner component within the outer component such that the inner component moves relative to the outer component.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

FIG. 1 is a schematic illustration of a substrate of a diagnostic device, according to some embodiments;

FIGS. 2A-2C are, according to some embodiments, schematic illustrations of an inner component and a substrate of a diagnostic device;

FIG. 3 is a schematic illustration of an outer casing of a diagnostic device, according to some embodiments;

FIGS. 4A-4C are, according to some embodiments, schematic illustrations of a diagnostic device comprising an outer casing, an inner movable member, and a substrate;

FIGS. 5A-5B are schematic illustrations of a diagnostic device comprising two safety clips, according to some embodiments;

FIG. 6 is, according to some embodiments, a schematic illustration of a diagnostic device comprising an inner component configured to screw into an outer component;

FIGS. 7A-7C are schematic illustrations of diagnostic testing kits, according to some embodiments; and

FIGS. 8A-8L are, according to some embodiments, schematic illustrations of steps of a diagnostic testing method.

### DETAILED DESCRIPTION

Described herein in an embodiment is a diagnostic device for detecting the presence of one or more target nucleic acids (e.g., a nucleic acid of a pathogen, such as SARS-CoV-2 or an influenza virus). In some cases, the diagnostic device comprises a substrate comprising both a reagent delivery region comprising one or more reagents (e.g., one or more nucleic acid amplification reagents) and a lateral flow assay region configured to detect one or more target nucleic acids. The substrate may be removably or permanently coupled to an inner component that is movable relative to an outer component. The diagnostic device may further comprise a sample-collecting component coupled to the outer component and/or the inner component. In some embodiments, the inner component may be moved relative to the outer component in order to sequentially expose the substrate to the collected sample. In some cases, the diagnostic device may be used with a reaction tube comprising one or more liquids (e.g., a reaction buffer) and/or a heating unit.

As the COVID-19 pandemic has highlighted, there is a critical need for rapid, accurate systems and methods for diagnosing diseases-particularly infectious diseases. In the absence of diagnostic testing, asymptomatic infected individuals may unknowingly spread the disease to others, and symptomatic infected individuals may not receive appropriate treatment. With testing, however, infected individuals may take appropriate precautions (e.g., self quarantine) to reduce the risk of infecting others and may receive targeted treatment as helpful.

While diagnostic tests for various diseases are known, such tests often require specialized knowledge of laboratory techniques and/or expensive laboratory equipment. For example, polymerase chain reaction (“PCR”) tests generally require skilled technicians and expensive, bulky thermocyclers. In addition, there remains a need for diagnostic tests that are both rapid and highly accurate. Known diagnostic tests with high levels of accuracy often take hours, or even days, to return results, while more rapid tests generally have low levels of accuracy. Additionally, many rapid diagnostic tests detect antibodies, which generally can only reveal whether a person has previously had a disease, not whether the person has an active infection. In contrast, nucleic acid tests (i.e., tests that detect one or more target nucleic acids) may indicate that a person has an active infection.

Diagnostic devices described herein may be easily operated by untrained individuals. In some cases, diagnostic devices described herein are operated using only basic motions (e.g., pushing one component into another component, rotating one component relative to another component). Unlike prior art diagnostic tests, some embodiments described herein may not require knowledge of even basic laboratory techniques (e.g., pipetting). Similarly, some embodiments described herein may not require expensive laboratory equipment (e.g., thermocyclers). Thus, even untrained individuals may properly operate diagnostic devices described herein.

In addition, diagnostic devices described herein may be safely operated by untrained individuals. In some cases, for example, reagents are contained within the diagnostic devices and/or a reaction tube, such that users are not exposed to any potentially harmful chemicals. In some cases, the diagnostic devices comprise a built-in sample-collecting component. After a sample has been collected, a user may not need to touch the sample-collecting component (or anything in its vicinity) when performing a diagnostic method using the diagnostic device, allowing the sample-collecting component to remain uncontaminated and reducing the risk that a user may be exposed to a pathogen.

Diagnostic devices described herein are also highly sensitive and accurate. In some embodiments, the diagnostic devices are configured to detect one or more target nucleic acids using nucleic acid amplification (e.g., an isothermal nucleic acid amplification method). Through nucleic acid amplification, the diagnostic devices are able to accurately detect the presence of extremely small amounts of a target nucleic acid.

As a result, the diagnostic devices may be useful in a wide variety of contexts. For example, in some cases, the devices may be available over the counter for use by consumers. In such cases, untrained consumers may be able to self administer the test (or administer the test to friends and family members) in their own homes (or any other location of their choosing). In some cases, the devices may be operated by employees or volunteers of an organization (e.g., a school, a medical office, a business). For example, a school (e.g., an elementary school, a high school, a university) may test its students, teachers, and/or administrators, a medical office (e.g., a doctor's office, a dentist's office) may test its patients and/or health care providers, or a business may test its employees for a particular disease. In each case, the diagnostic devices may be operated by the subjects of the tests (e.g., students, teachers, patients, employees) or by designated individuals (e.g., a school nurse, a teacher, a school administrator, a receptionist). Point-of-care administration is also contemplated herein, where the diagnostic devices are operated by a trained medical professional in a point-of-care setting.

In some embodiments, the diagnostic devices are relatively small. In certain cases, for example, a diagnostic device is approximately the size of a pen or a marker. Thus, unlike diagnostic tests that require bulky equipment, diagnostic devices described herein may be easily transported and/or easily stored in homes and businesses. In some embodiments, the diagnostic devices are relatively inexpensive. Since no expensive laboratory equipment (e.g., a thermocycler) is required, diagnostic devices described herein may be more cost effective than known diagnostic tests.

In some embodiments, any reagents contained within a diagnostic device described herein may be thermostabilized, and the diagnostic device may be shelf stable for a relatively long period of time. In certain embodiments, for example, the diagnostic device may be stored at approximately room temperature (e.g., 20° C. to 25° C.) for a relatively long period of time (e.g., at least 1 month, at least 3 months, at least 6 months, at least 9 months, at least 1 year) with no loss of activity or sensitivity.

### Overview of Diagnostic Device

According to some embodiments, a diagnostic device is configured to detect the presence or absence of one or more target nucleic acids in a sample. In certain cases, the one or more target nucleic acids comprise a nucleic acid of a pathogen (e.g., a viral, bacterial, fungal, protozoan, parasitic, or other pathogen). In some instances, the pathogen is severe acute respiratory syndrome coronavirus 2 (SARS-CoV-2), which causes coronavirus disease 2019 (COVID-19), or a variant thereof. In some instances, the pathogen is an influenza virus. The influenza virus may be an influenza A virus (e.g., HIN1, H3N2) or an influenza B virus. In certain instances, the diagnostic device is configured to detect the presence or absence of SARS-CoV-2 in samples (e.g., anterior nares specimens, saliva specimens, cell scrapings) collected from subjects (e.g., human subjects, animal subjects). In such instances, a positive result may indicate an active infection with COVID-19. However, the diagnostic devices described herein are not limited to detection of SARS-CoV-2 and, as discussed in further detail below, may be configured to detect a variety of other target nucleic acids.

In some embodiments, a diagnostic device comprises an outer casing and an inner member that is movable within the outer casing. In some cases, motions of the inner member relative to the outer casing (e.g., pushing the inner member into the outer casing, rotating the inner member relative to the outer casing) sequentially expose portions of the inner member to samples and/or reagents.

In certain embodiments, the diagnostic device comprises a sample-collecting component that is coupled to the outer casing and/or the inner member. The sample-collecting component may be used to collect a sample (e.g., a nasal secretion, an oral secretion, a genital secretion, a cell scraping, blood, urine) from a subject (e.g., a human subject, an animal subject). As one example, the sample-collecting component may comprise a swab element, and the swab element may be inserted into a cavity of a subject. In some embodiments, the cavity is a nasal cavity, an oral cavity, a vaginal cavity, an anal cavity, an ear canal, or another bodily orifice. In certain instances, the swab element may be used to collect a sample from the anterior nares of a subject. In some embodiments, the swab element may be used to collect one or more types of bodily fluids (e.g., bodily secretions).

In some embodiments, after the sample has been collected, the swab element may be inserted into a reaction tube comprising one or more liquids (e.g., a reaction buffer). A user may then perform one or more actions (also referred to as movements) that move the inner member relative to the outer casing. In some cases, for example, a first action (e.g., pushing or pulling the inner member relative to the outer casing, rotating the inner member relative to the outer casing) exposes a reagent delivery region of a substrate associated with the inner member to the sample and the one or more liquids (e.g., reaction buffer). In certain cases, this may cause one or more reagents in the reagent delivery region (e.g., lysis reagents, reverse transcription reagents, nucleic acid amplification reagents, CRISPR/Cas detection reagents) to be dissolved in the one or more liquids (e.g., reaction buffer). In some cases, one or more sequences from any target nucleic acids and/or control nucleic acids that are present in the sample may be amplified. A user may then perform a second action (e.g., pushing or pulling the inner member relative to the outer casing, rotating the inner member relative to the outer casing) that exposes a lateral flow assay region of the substrate to the fluidic contents of the reaction tube, which may now comprise amplified nucleic acids. The lateral flow assay region may then indicate the presence of any target nucleic acids and/or control nucleic acids (e.g., by the presence of one or more lines or other marks on the substrate).

Thus, the inner movable member of the diagnostic device may allow even an untrained individual to perform nucleic acid amplification using only basic motions (e.g., pushing, pulling, rotating). In addition, the built-in sample-collecting component may allow the untrained individual to collect a sample and amplify nucleic acids without contaminating the sample or being exposed to chemicals. In this manner, even an untrained individual can perform a highly sensitive diagnostic test that can rapidly and accurately detect the presence of target nucleic acids.

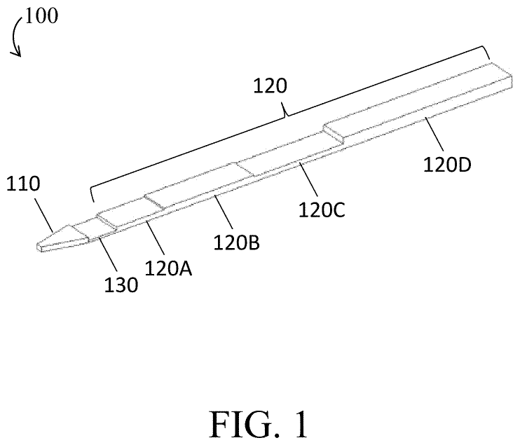

In some embodiments, a diagnostic device comprises a substrate. An exemplary substrate is shown in FIG. 1. In certain embodiments, the substrate comprises a reagent delivery region and a lateral flow assay region. For example, in FIG. 1, substrate 100 comprises reagent delivery region 110 and lateral flow assay region 120.

In some embodiments, reagent delivery region 110 comprises one or more layers comprising one or more materials that allow fluid transport (e.g., via capillary action). Non-limiting examples of suitable materials include polyethersulfone, cellulose, polycarbonate, nitrocellulose, sintered polyethylene, and glass fibers. In some embodiments, reagent delivery region 110 comprises one or more reagents. In some cases, at least one of the one or more reagents is thermostabilized (e.g., lyophilized, crystallized, air jetted, dried). In some cases, all of the one or more reagents are thermostabilized. In some embodiments, the one or more reagents comprise one or more lysis reagents (e.g., enzymes, detergents) and/or one or more reverse transcription reagents (e.g., reverse transcriptase). In certain embodiments, the one or more reagents comprise one or more nucleic acid amplification reagents. Non-limiting examples of suitable nucleic acid amplification reagents include reagents for loop-mediated isothermal amplification (“LAMP”), recombinase polymerase amplification (“RPA”), thermophilic helicase dependent amplification (“tHDA”), nucleic acid sequence-based amplification (“NASBA”), and/or nicking enzyme amplification reaction (“NEAR”). In certain embodiments, the nucleic acid amplification reagents comprise PCR reagents. In some embodiments, the one or more reagents comprise one or more CRISPR/Cas detection reagents.

In some embodiments, lateral flow assay region 120 is configured to detect one or more target nucleic acids. In certain embodiments, lateral flow assay region 120 comprises one or more layers comprising one or more materials that that allow fluid transport (e.g., via capillary action). Non-limiting examples of suitable materials include polyethersulfone, cellulose, polycarbonate, nitrocellulose, sintered polyethylene, and glass fibers. The one or more fluid-transporting materials of lateral flow assay region 120 may be the same as or different from the one or more fluid-transporting materials of reagent delivery region 110.

In some cases, lateral flow assay region 120 comprises one or more test lines, where each test line is configured to detect a target nucleic acid. In some embodiments, each of the one or more test lines comprises one or more capture reagents (e.g., immobilized antibodies). In some cases, lateral flow assay region 120 further comprises one or more control lines. In certain instances, at least one control line is a human (or animal) nucleic acid control line that, if detectable, confirms that a human (or animal) sample was properly collected and processed. In certain instances, at least one control line is a lateral flow control line that, if detectable, confirms that a liquid reached the lateral flow assay region.

In certain embodiments, lateral flow assay region 120 comprises two or more sub-regions. For example, in FIG. 1, lateral flow assay region 120 comprises sample pad 120A (e.g., where a liquid sample is introduced to lateral flow assay region 120), particle conjugate pad 120B (e.g., where labeled nanoparticles may be located), test pad 120C (e.g., where the one or more test lines and/or control lines may be located), and wicking area 120D. In some cases, wicking area 120D may allow sufficient fluid to flow along the device. In some embodiments, a lateral flow assay region comprises a single region.

In some embodiments, a reagent delivery region may be separated from a lateral flow assay region by a separation region. In FIG. 1, substrate 100 comprises separation region 130 positioned between reagent delivery region 110 and lateral flow assay region 120. In some embodiments, separation region 130 comprises one or more non-wicking materials and does not comprise any materials that allow fluid transport (e.g., via capillary action). A “non-wicking material” generally refers to a material that does not allow fluid transport (e.g., via capillary action). In some cases, the non-wicking material is a substantially non-porous material. Examples of suitable non-wicking materials include, but are not limited to, polymers (e.g., polyethylene terephthalate, polyethylene naphthalate, polyvinyl chloride, polyurethane), metals, metal alloys, and ceramics.

In some embodiments, a substrate may be associated with an inner component. In certain cases, for example, an inner component may at least partially enclose at least a portion of a substrate. In some instances, a substrate may be removably coupled to an inner component. In some instances, a substrate may be permanently attached to (e.g., integrally formed with) an inner component.

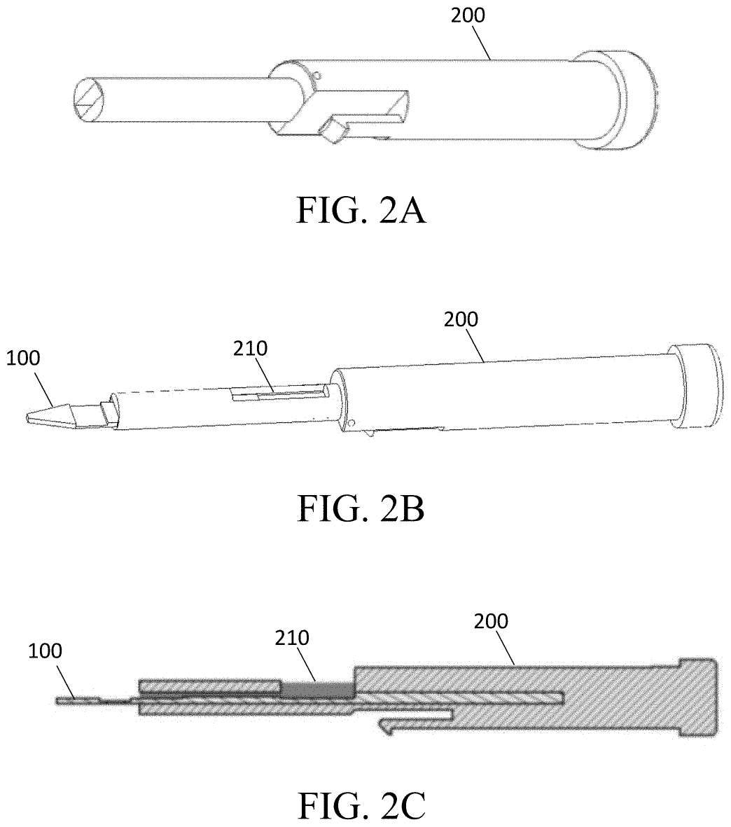

As one example, FIGS. 2A-2C show an exemplary inner component associated with a substrate, according to some embodiments. FIG. 2A shows an external view of inner component 200, and FIG. 2B shows an external view of substrate 100 positioned within inner component 200. As shown in FIG. 2B, inner component 200 may comprise an opening 210 through which at least a portion of substrate 100 is visible. FIG. 2C shows a cross-sectional view of substrate 100 positioned within inner component 200.

As discussed in further detail below, inner component 200 may be formed from any suitable material. In some embodiments, inner component 200 comprises a thermoplastic polymer and/or a metal. Inner component 200 may be formed by injection molding, an additive manufacturing process (e.g., 3D printing), and/or a subtractive manufacturing process (e.g., laser cutting).

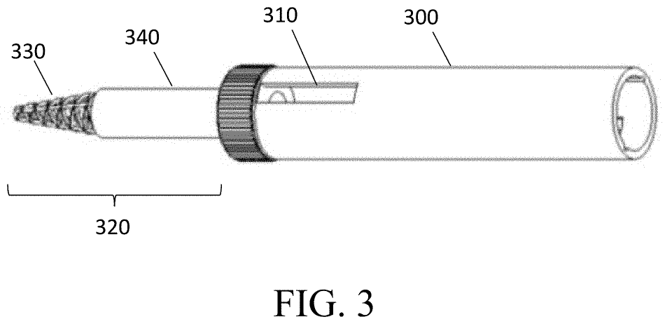

In some embodiments, a substrate and an inner component may be associated with an outer component. An illustrative embodiment of an outer component is shown in FIG. 3. In FIG. 3, outer component 300 comprises an outer casing having an opening 310. Like inner component 200, outer component 300 may be formed from any suitable material. In some embodiments, outer component 300 comprises a thermoplastic polymer and/or a metal. Outer component 300 may be formed by injection molding, an additive manufacturing process (e.g., 3D printing), and/or a subtractive manufacturing process (e.g., laser cutting).

In some embodiments, outer component 300 is coupled to sample-collecting component 320. A sample-collecting component may be removably or permanently coupled to either an outer component or an inner component of a diagnostic device. In FIG. 3, sample-collecting component 320 comprises swab element 330 and stem element 340. In certain instances, swab element 330 comprises an absorbent material. Examples of suitable absorbent materials include, but are not limited to, cotton, polyester, polyurethane, rayon, nylon, microfiber, viscose, cellulose, and alginate. In some instances, swab element 330 is a foam swab or a flocked swab (e.g., comprising flocked fibers of a material). In some instances, swab element 330 comprises a thermoplastic polymer and/or a metal. In some such instances, swab element 330 may be formed by injection molding, an additive manufacturing process (e.g., 3D printing), and/or a subtractive manufacturing process (e.g., laser cutting).

As shown in FIG. 3, stem element 340 of sample-collecting component 320 may have a smaller maximum diameter than the maximum diameter of outer component 300. In some cases, sample-collecting component 320 may be used for collection of a sample from a subject (e.g., a human subject, an animal subject). In certain cases, for example, sample-collecting component 320 may be configured to be inserted into a cavity (e.g., a nasal cavity, an oral cavity, a vaginal cavity, an anal cavity, an ear canal) of the subject. In some cases, a stem element having a relatively small diameter may facilitate insertion into a cavity of a subject.

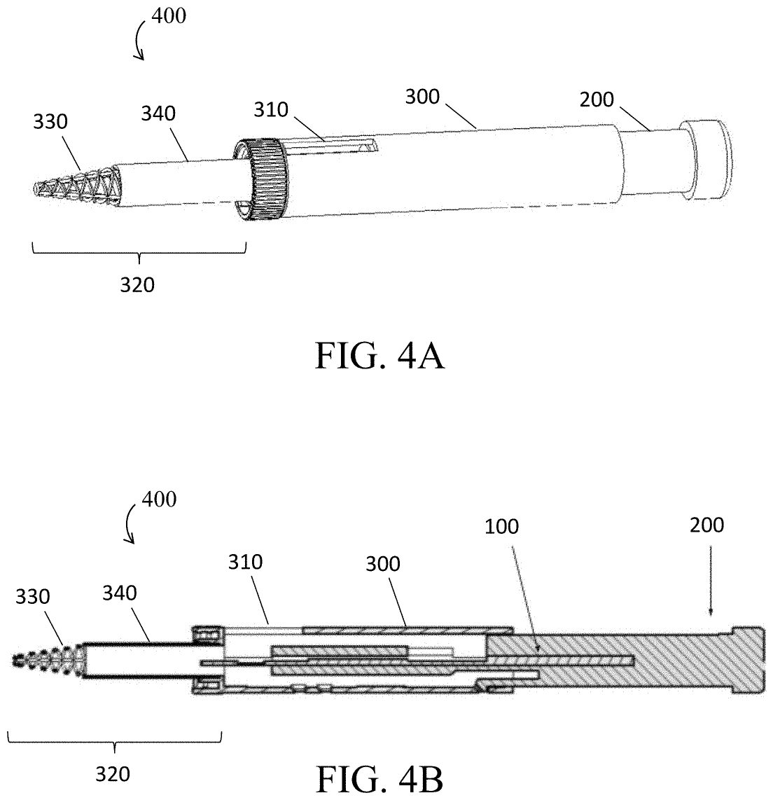

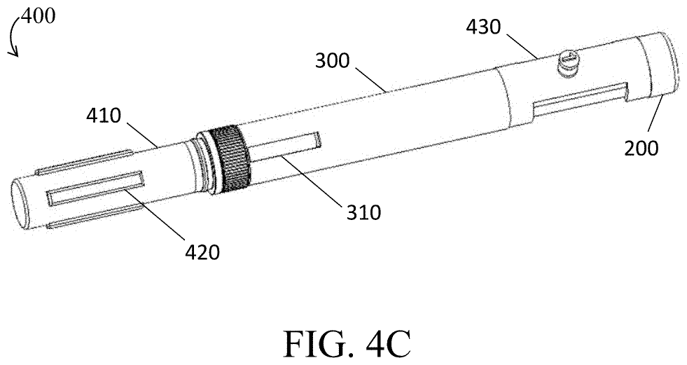

According to some embodiments, a diagnostic device comprises an outer component, an inner component, and a substrate. In certain cases, the inner component and the substrate may be movable relative to the outer component. An illustrative embodiment is shown in FIGS. 4A-4C. In FIG. 4A, diagnostic device 400 comprises inner component 200 and outer component 300. In FIG. 4A, inner component 200 is movable (e.g., by pushing and/or pulling) relative to outer component 300. In particular, at least a portion of inner component 200 has a maximum diameter that is less than a maximum diameter of outer component 300 (e.g., the outer casing of outer component 300), and at least a portion of inner component 200 may be inserted into at least a portion of outer component 300. From the cross-sectional view of diagnostic device 400 shown in FIG. 4B, it can be seen that inner component 200 is coupled to substrate 100.

In some embodiments, a diagnostic device further comprises a removable cap. In FIG. 4C, removable cap 410 covers sample-collecting component 320 (e.g., swab element 330 and stem element 340). In some cases, a removable cap may cover only a portion of a sample-collecting component (e.g., a swab element). In certain embodiments, the removable cap may comprise one or more protruding elements. In FIG. 4C, for example, removable cap 410 comprises a plurality of protruding elements 420. In some cases, the one or more protruding elements may prevent the removable cap from being inserted into a reaction tube or a heating unit.

In some embodiments, a diagnostic device further comprises a safety clip configured to prevent movement of an inner component relative to an outer component until the safety clip is removed. In FIG. 4C, diagnostic device 400 comprises safety clip 430, which prevents inner component 200 from moving relative to outer component 300. When safety clip 430 is removed, inner component 200 can be pushed a first distance into outer component 300.

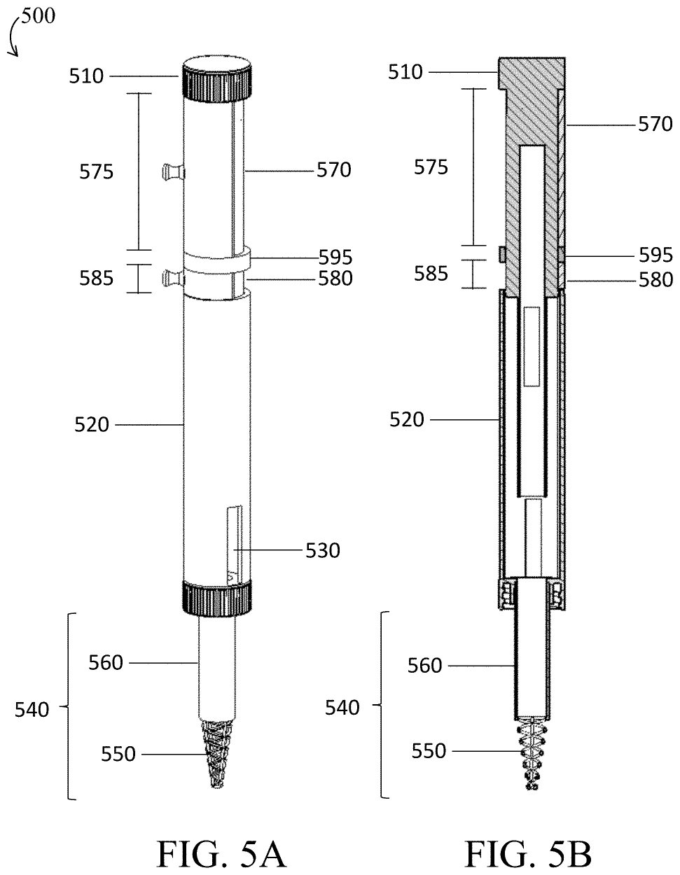

In some embodiments, a diagnostic device further comprises one or more additional safety clips configured to prevent movement of an inner component relative to an outer component until the one or more additional safety clips are removed. As one example, FIG. 5A shows an external view of diagnostic device 500 comprising first safety clip 570 and second safety clip 580. As shown in FIG. 5A, diagnostic device 500 comprises inner component 510, outer component 520, and sample-collecting component 540. In FIG. 5A, sample-collecting component 540 comprises swab element 550 and stem element 560. As further shown in FIG. 5A, outer component 520 comprises an opening 530 through which at least a portion of any internal components may be visible. In some cases, first safety clip 570 and second safety clip 580 may prevent inner component 510 from moving relative to outer component 520. Removal of first safety clip 570 may allow inner component 510 to move first distance 575. Removal of second safety clip 580 may allow inner component 510 to move second distance 585. This is further visible in FIG. 5B, which shows a cross-sectional view of diagnostic device 500. In some embodiments, first safety clip 570 and second safety clip 580 are separated by one or more components. In certain instances, for example, first safety clip 570 and second safety clip 580 are separated by washer 595 (e.g., a sliding washer). In some cases, the presence of a component between first safety clip 570 and second safety clip 580 may prevent friction between the safety clips. In some instances, this may prevent removal of first safety clip 570 from also removing second safety clip 580.

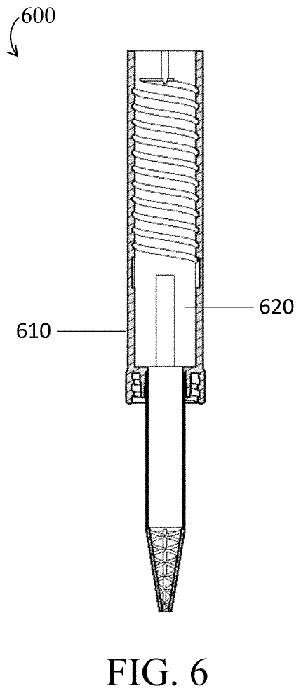

In some embodiments, a diagnostic device comprises an inner component configured to be screwed into an outer component. FIG. 6 shows a cross-sectional view of a portion of diagnostic device 600 comprising outer component 610 and inner component 620, where inner component 620 comprises a plurality of screw threads and outer component 610 comprises a plurality of matching screw threads such that inner component 620 can be screwed into outer component 610.

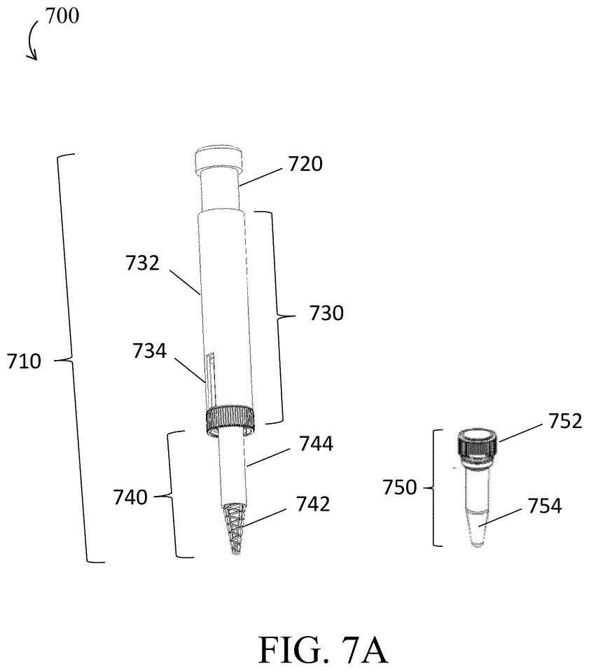

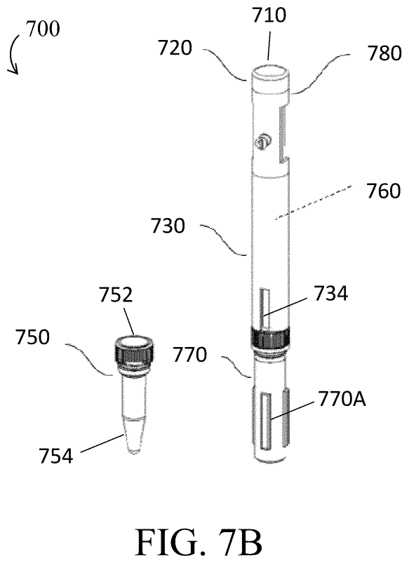

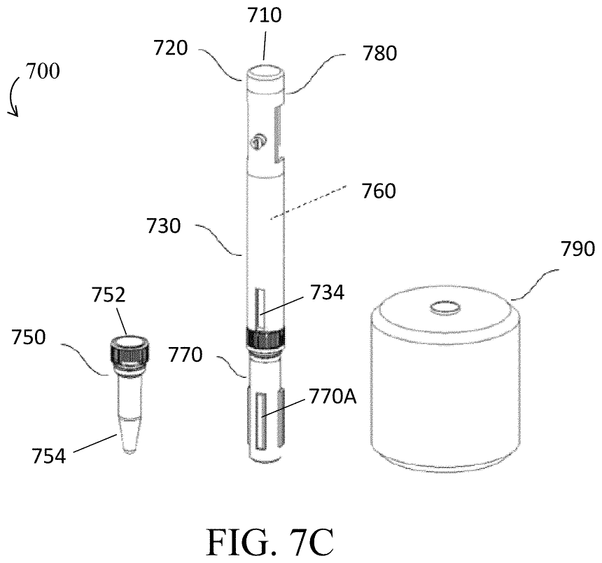

Some embodiments are directed to a diagnostic test kit. An illustrative embodiment of a diagnostic test kit is shown in FIGS. 7A-7B. In FIG. 7A, diagnostic test kit 700 comprises diagnostic device 710 and reaction tube 750. Diagnostic device 710 comprises inner component 720, outer component 730, and sample-collecting component 740. Outer component 730 comprises outer casing 732 and opening 734 in outer casing 732. Sample-collecting component 740 comprises swab element 742 and stem element 744. In addition to diagnostic device 710, diagnostic test kit 700 further comprises reaction tube 750. As shown in FIG. 7A, reaction tube 750 comprises cap 752 and fluidic contents 754. In some embodiments, fluidic contents 754 comprise one or more liquids. In certain embodiments, the one or more liquids comprise a reaction buffer. In certain instances, the reaction buffer comprises one or more buffers. Non-limiting examples of suitable buffers include phosphate-buffered saline (“PBS”) and Tris. In certain instances, the reaction buffer comprises one or more salts. Non-limiting examples of suitable salts include magnesium sulfate, magnesium acetate tetrahydrate, potassium acetate, potassium chloride, and ammonium sulfate. As discussed in further detail below, the reaction buffer may have any suitable pH. Further, fluidic contents 754 of reaction tube 750 may vary over time; as a diagnostic test method using the diagnostic test kit is performed, fluidic contents 754 may, at times, comprise a reaction buffer, lysed cells, complementary DNA (cDNA), and/or amplified nucleic acids (i.e., amplicons).

In operation, cap 752 of reaction tube 750 may be removed, exposing fluidic contents 754. In some embodiments, sample-collecting component 740 is used to collect a sample (e.g., nasal secretion, oral secretion, genital secretion, cell scraping, blood, urine) from a subject (e.g., a human subject, an animal subject). In some instances, for example, swab element 742 is inserted into a cavity (e.g., a nasal cavity, an oral cavity, a vaginal cavity, an anal cavity, an ear canal) of the subject to collect the sample. Sample-collecting component 740, bearing the sample, is then inserted into fluidic contents 754 of reaction tube 750. In some embodiments, outer component 730 may be secured to reaction tube 750 (e.g., by a screw, a snap locking mechanism, or other fastener). A first action (e.g., pushing inner component 720 into outer component 730, rotating inner component 720 relative to outer component 730) is performed that moves inner component 720 relative to outer component 730 such that a first portion of inner component 720 is in physical contact with fluidic contents 754 of reaction tube 750. In some cases, a second action (e.g., pushing inner component 720 into outer component 730, rotating inner component 720 relative to outer component 730) is performed that further moves inner component 720 relative to outer component 730 such that a second portion of inner component 720 is in physical contact with fluidic contents 754 of reaction tube 750. In certain embodiments, one or more additional actions moving inner component 720 relative to outer component 730 may be performed. In some cases, an indicator of the presence or absence of a target nucleic acid may be detectable through opening 734 of outer casing 732.

In some cases, the first portion of inner component 720 is a reagent delivery region of a substrate associated with inner component 720. In certain cases, the reagent delivery region comprises one or more reagents (e.g., lysis reagents, reverse transcription reagents, nucleic acid amplification reagents, CRISPR/Cas detection reagents). In some instances, the one or more reagents are thermostabilized. In certain instances, contact between the first portion of inner component 720 (e.g., the reagent delivery region of the substrate) and fluidic contents 754 of reaction tube 750 causes the one or more reagents to be dissolved into fluidic contents 754.

In some cases, the second portion of inner component 720 is a lateral flow assay region of a substrate. In some embodiments, the reagent delivery region and the lateral flow assay region of the substrate are separated by a separation region. In certain embodiments, the separation region of the substrate comprises one or more non-wicking materials (e.g., materials that do not allow fluid transport via capillary action) and does not contain any materials that allow fluid transport (e.g., via capillary action).

In some embodiments, the first action moving inner component 720 relative to outer component 730 causes the reaction delivery region of the substrate to physically contact fluidic contents 754 of reaction tube 750 but does not cause the lateral flow assay region of the substrate to physically contact fluidic contents 754. In some cases, the separation region between the reagent delivery region and the lateral flow assay region prevents liquid from being transported from the reagent delivery region to the lateral flow assay region (e.g., via capillary action) after the first action is performed and before the second action is performed.

In some embodiments, the second action moving inner component 720 relative to outer component 730 causes the lateral flow assay region of the substrate to physically contact fluidic contents 754 of reaction tube 750. In some cases, fluidic contents 754 comprise one or more amplified nucleic acids (i.e., amplicons) prior to the second action being performed. In certain embodiments, the second action causes at least a portion of fluidic contents 754 (e.g., containing one or more amplicons) to be transported through the lateral flow assay region of the substrate via capillary action.

In certain embodiments, a diagnostic test kit comprises a diagnostic device comprising a removable cap and/or a safety clip. In FIG. 7B, for example, diagnostic device 710 comprises removable cap 770 covering at least a portion of sample-collecting component 740. Removable cap 770 may comprise one or more protruding elements 770A. As shown in FIG. 7B, diagnostic device 710 may further comprise first safety clip 780, which may prevent motion of inner component 720 relative to outer component 730 (i.e., maintain inner component 720 and outer component 730 in a particular configuration) until first safety clip 780 is removed.

In operation, removable cap 770 may be removed from sample-collecting component 740 prior to collecting a sample. In certain embodiments, removable cap 770 may be used to hold reaction tube 750 (e.g., during sample collection). In some cases, after a sample has been collected and the sample-collecting component has been inserted into reaction tube 750, first safety clip 780 may be removed to allow a first motion of inner component 720 relative to outer component 730 (e.g., inner component 720 may be pushed a first distance into outer component 730). In certain embodiments, inner component 720 may be further moved relative to outer component 730.

In some embodiments, a diagnostic test kit further comprises a heating unit. In FIG. 7C, diagnostic test kit 700 comprises diagnostic device 710, reaction tube 750, and heating unit 790. Diagnostic device 710 comprises inner component 720, outer component 730, and substrate 760. Reaction tube 750 comprises cap 752 and fluidic contents 754. Heating unit 790 may be any device capable of heating fluidic contents 754 of reaction tube 750 to one or more desired temperatures for a desired time.

In operation, cap 752 of reaction tube 750 may be removed, exposing fluidic contents 754. Reaction tube 750 may then be placed in heater 790. Removable cap 770 of diagnostic device 710 may be removed, exposing sample-collecting component 740 of diagnostic device 710 (not shown in FIG. 7C). One or more protruding elements 770A on removable cap 770 may prevent removable cap 770 from mistakenly being inserted into reaction tube 750 and/or heating unit 790. In some embodiments, sample-collecting component 740 may be used to collect a sample (e.g., nasal secretion, oral secretion, genital secretion, cell scraping, blood, urine) from a subject (e.g., a human subject, an animal subject). In some instances, for example, swab element 742 may be inserted into a cavity (e.g., a nasal cavity, an oral cavity, a vaginal cavity, an anal cavity, an ear canal) of the subject to collect the sample. Sample-collecting component 740, bearing the sample, may then be inserted into fluidic contents 754 of reaction tube 750. In some embodiments, first safety clip 780 may be removed, and a first action (e.g., pushing inner component 720 into outer component 730, rotating inner component 720 relative to outer component 730) may be performed that moves inner component 720 relative to outer component 730 such that a first portion of inner component 720 is in physical contact with fluidic contents 754 of reaction tube 750. In certain embodiments, the first portion of inner component 720 (e.g., a reagent delivery region of substrate 760) comprises one or more reagents (e.g., lysis reagents, reverse transcription reagents, nucleic acid amplification reagents, CRISPR/Cas detection reagents). In some instances, the one or more reagents are thermostabilized. In some embodiments, physical contact between the first portion of inner component 720 and fluidic contents 754 of reaction tube 750 causes the one or more reagents to be dissolved in fluidic contents 754 of reaction tube 750.

In some embodiments, heating unit 790 heats fluidic contents 754 of reaction tube 750 to one or more desired temperatures for a desired amount of time. In certain instances, heating fluidic contents 754 of reaction tube 750 may facilitate lysis of cells in the sample (e.g., via thermal or chemical lysis). In certain instances, heating fluidic contents 754 may facilitate reverse transcription of RNA in the sample (e.g., viral RNA) to DNA (e.g., cDNA). In certain instances, heating fluidic contents 754 may facilitate amplification of nucleic acids (e.g., via LAMP, RPA, tHDA, NASBA, or NEAR). As a result, after heating, fluidic contents 754 may comprise amplified nucleic acids (i.e., amplicons).

In some embodiments, a second action (e.g., pushing inner component 720 into outer component 730, rotating inner component 720 relative to outer component 730) is performed that further moves inner component 720 relative to outer component 730 such that a second portion of inner component 720 is in physical contact with fluidic contents 754 of reaction tube 750. In some embodiments, the second portion of inner component 720 is a lateral flow assay region of substrate 760. In some embodiments, the second action may cause at least a portion of fluidic contents 754 (e.g., including amplicons) to be transported onto at least a portion of the lateral flow assay region of substrate 760 (e.g., via capillary action). In certain embodiments, the second action may align an opening in inner component 720 with opening 734 in outer component 730. In some cases, an indicator of the presence or absence of a target nucleic acid may be detectable through opening 734 of outer casing 732. In some instances, the indicator may be the presence of one or more lines or other detectable markings.

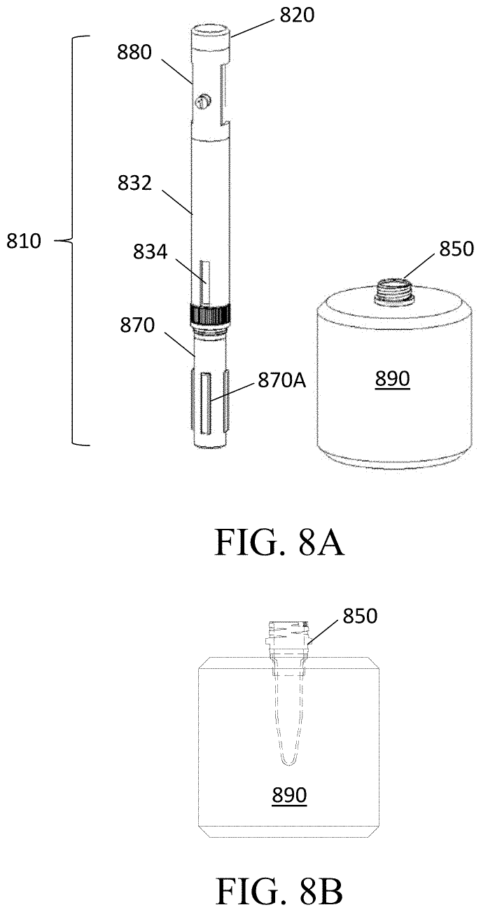

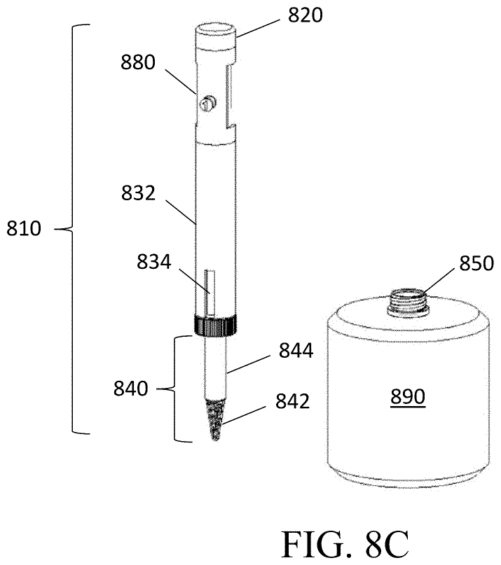

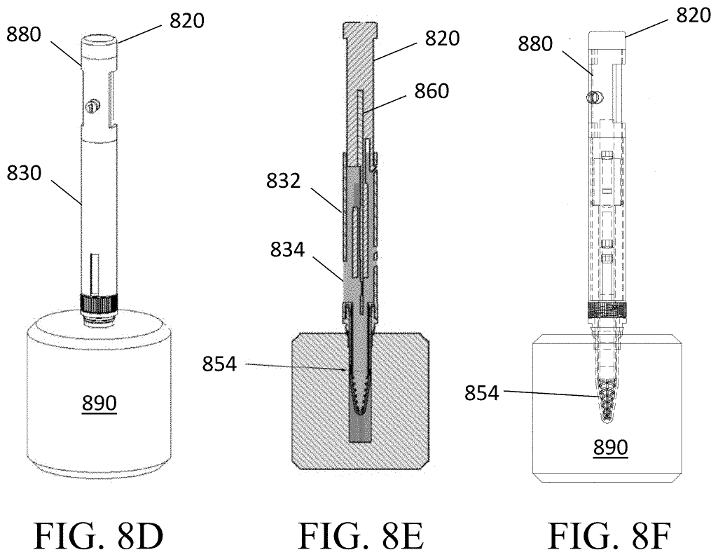

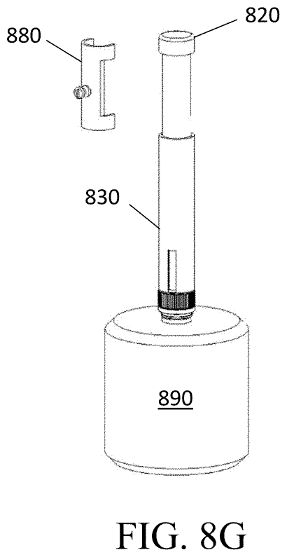

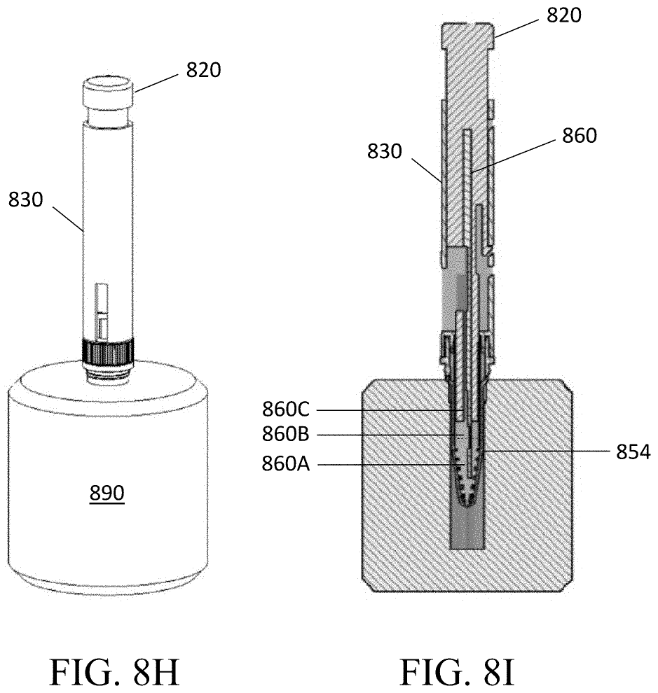

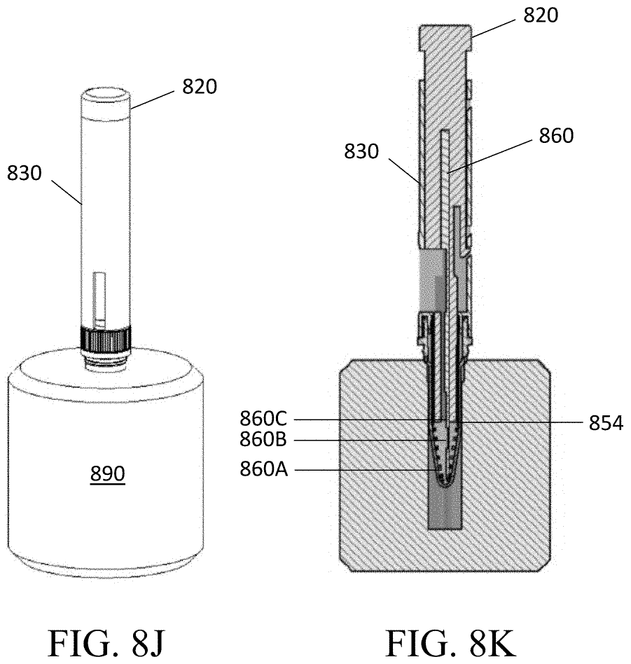

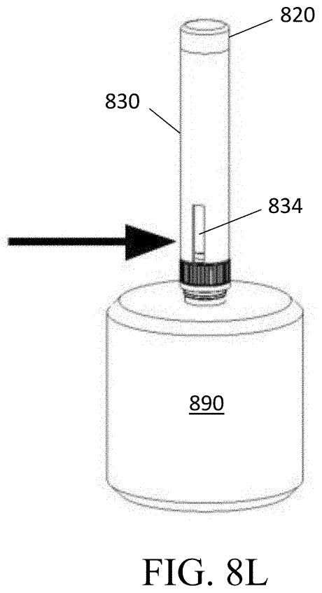

Some embodiments are directed to a diagnostic testing method. One embodiment of a diagnostic testing method is shown in FIGS. 8A-8L. As shown in FIG. 8A, a user may remove a cap of reaction tube 850 and place reaction tube 850 in heating unit 890. One or more protruding elements 870A on removable cap 870 of diagnostic device 810 may prevent removable cap 870 from being mistakenly inserted into reaction tube 850 and/or heating unit 890. FIG. 8B shows a cross-sectional view of reaction tube 850 in heating unit 890.

As shown in FIG. 8C, a user may remove cap 870 from diagnostic device 810, exposing sample-collecting component 840, which comprises swab element 842 and stem element 844. In some embodiments, cap 870 may be configured to hold reaction tube 850 (e.g., during sample collection, prior to placing reaction tube 850 in heating unit 890). Sample-collecting component 840 may then be used to collect a sample (e.g., collecting a nasal secretion, oral secretion, genital secretion, cell scraping, blood, or urine). In some embodiments, the sample is collected by inserting at least a portion of sample-collecting component 840 into a cavity of the subject.

As shown in FIGS. 8D-8F, after a sample has been collected, diagnostic device 810 may be inserted into reaction tube 850 in heating unit 890 such that at least a portion of swab element 842 is in physical contact with fluidic contents 854 of reaction tube 850. FIG. 8D shows an external view, and FIGS. 8E-8F show cross-sectional views, of diagnostic device 810 inserted into reaction tube 850, which is in heating unit 890. FIGS. 8E and 8F show that at least a portion of swab element 842 of sample-collecting component 840 is in physical contact with fluidic contents 854 of reaction tube 850. In addition, FIGS. 8E and 8F show that substrate 860 is not in physical contact with fluidic contents 854 of reaction tube 850. In certain embodiments, diagnostic device 810 may be screwed into or otherwise fastened to reaction tube 850 and/or heating unit 890 to provide a more secure connection.

As shown in FIG. 8G, safety clip 880 may be removed from diagnostic device 810. The removal of safety clip 880 may allow inner component 820 to move relative to outer component 830.

As shown in FIGS. 8H and 8I, one or more motions may be performed to move inner component 820 relative to outer component 830. The one or more motions may comprise pushing inner component 820 a first distance into outer component 830. FIG. 8H shows an external view of diagnostic device 810 and heating unit 890 after inner component 820 has been pushed a first distance into outer component 830. In some embodiments, the one or more motions result in a first portion of substrate 860 (e.g., reagent delivery region 860A) contacting fluidic contents 854 of reaction tube 850. FIG. 8I shows a cross-sectional view of diagnostic device 810 after inner component 820 has been moved relative to outer component 830 such that reagent delivery region 860A is in contact with fluidic contents 854 of reaction tube 850. As a result, fluidic contents 854 of reaction tube 850 may comprise one or more reagents (e.g., lysis reagents, reverse transcription reagents, nucleic acid amplification reagents, CRISPR/Cas detection reagents) dissolved in one or more liquids (e.g., a reaction buffer). As further shown in FIG. 8I, substrate 860 further comprises separation region 860B and lateral flow assay region 860C. Lateral flow assay region 860C is not in physical contact with fluidic contents 854 of reaction tube 850, and separation region 860B prevents any liquids from being transported to lateral flow assay region 860C.

In some embodiments, heating unit 890 may heat fluidic contents 854 of reaction tube 850 to one or more desired temperatures for a desired amount of time. In certain instances, heating fluidic contents 854 of reaction tube 850 may facilitate lysis of cells in the sample (e.g., via thermal or chemical lysis). In certain instances, heating fluidic contents 854 may facilitate reverse transcription of RNA in the sample (e.g., viral RNA) to DNA (e.g., cDNA). In certain instances, heating fluidic contents 854 may facilitate amplification of nucleic acids (e.g., via LAMP, RPA, tHDA, NASBA, or NEAR). As a result, after heating, fluidic contents 854 may comprise amplified nucleic acids (i.e., amplicons).

As shown in FIGS. 8J and 8K, one or more additional motions may be performed to further move inner component 820 relative to outer component 830. The one or more additional motions may comprise further pushing inner component 820 a second distance into outer component 830. FIG. 8J shows an external view of diagnostic device 810 and heating unit 890 after inner component 820 has been pushed a second distance into outer component 830. In some embodiments, the one or more additional motions result in a second portion of substrate 860 (e.g., lateral flow assay region 860C) contacting fluidic contents 854 of reaction tube 850. FIG. 8K shows a cross-sectional view of diagnostic device 810 after inner component 820 has been moved relative to outer component 830 such that at least a portion of lateral flow assay region 860C is in contact with fluidic contents 854 of reaction tube 850. From FIG. 8K, it can be seen that reagent delivery region 860A, separation region 860B, and lateral flow assay region 860C are all in physical contact with fluidic contents 854 of reaction tube 850 after the one or more additional motions have been performed. In some cases, contact between lateral flow assay region 860C and fluidic contents 854 may allow amplicons in fluidic contents 854 to travel via capillary action through lateral flow assay region 860C, which may comprise one or more test lines comprising one or more capture reagents (e.g., immobilized antibodies) configured to detect one or more target nucleic acids. In some cases, lateral flow assay region 860C may further comprise one or more control lines comprising a human nucleic acid control and/or a lateral flow control. In some cases, if the one or more target nucleic acids are present in the sample, the one or more test lines will become detectable. In some cases, if a sample has been properly collected and/or the diagnostic test has been properly run, the one or more control lines will become detectable. As shown in FIG. 8L, the one or more lines on substrate 860 (e.g., in lateral flow assay region 860C) may be detectable when opening 834 in outer component 830 is aligned with an opening in inner component 820.

### Overall Characteristics of Diagnostic Device

In some embodiments, a diagnostic device is configured to detect a first target nucleic acid. In some cases, the first target nucleic acid is a nucleic acid of a pathogen. The pathogen may be a viral, bacterial, fungal, protozoan, parasitic, or other pathogen. In some embodiments, the pathogen is a respiratory pathogen or a sexually transmitted pathogen.

In some embodiments, the pathogen is a viral pathogen. Non-limiting examples of viral pathogens include coronaviruses, influenza viruses, rhinoviruses, parainfluenza viruses (e.g., parainfluenza 1-4), enteroviruses, adenoviruses, respiratory syncytial viruses, and metapneumoviruses. In certain embodiments, the viral pathogen is SARS-CoV-2. In some embodiments, the viral pathogen is a variant of SARS-CoV-2. In certain instances, the SARS-CoV-2 variant is SARS-CoV-2 D614G, a SARS-CoV-2 variant of B.1.1.7 lineage (e.g., 20B/501Y.V1 Variant of Concern (VOC) 202012/01), a SARS-CoV-2 variant of B.1.351 lineage (e.g., 20C/501Y.V2), a SARS-CoV-2 variant of B.1.427 lineage, a SARS-CoV-2 variant of B.1.429 lineage, or a SARS-CoV-2 variant of B.1.617.2 lineage. In certain instances, the SARS-CoV-2 variant comprises one or more mutations selected from the group consisting of D614G (i.e., a mutation of the 6140 amino acid from aspartic acid (D) to glycine (G)), A222V, N501Y, E484K, K417N, and K417T. In certain embodiments, the viral pathogen is an influenza virus, where the influenza virus is an influenza A virus (e.g., H1N1, H3N2) or an influenza B virus.

Other viral pathogens include, but are not limited to, adenovirus; Herpes simplex virus, type 1; Herpes simplex virus, type 2; encephalitis virus; papillomavirus (e.g., human papillomavirus); Varicella zoster virus; Epstein-Barr virus; human cytomegalovirus; human herpesvirus, type 8; BK virus; JC virus; smallpox; polio virus; hepatitis A virus; hepatitis B virus; hepatitis C virus; hepatitis D virus; hepatitis E virus; human immunodeficiency virus (HIV); human bocavirus; parvovirus B19; human astrovirus; Norwalk virus; coxsackievirus; rhinovirus; yellow fever virus; dengue virus; West Nile virus; Guanarito virus; Junin virus; Lassa virus; Machupo virus; Sabia virus; Crimean-Congo hemorrhagic fever virus; Ebola virus; Marburg virus; measles virus; mumps virus; rubella virus; Hendra virus; Nipah virus; rabies virus; rotavirus; orbivirus; Coltivirus; Hantavirus; Middle East Respiratory Coronavirus; Zika virus; norovirus; Chikungunya virus; and Banna virus.

In some embodiments, the pathogen is a bacterial pathogen. The bacterial pathogen may be a Gram-positive bacterium or a Gram-negative bacterium. Bacterial pathogens include, but are not limited to, Acinetobacter baumannii, Bacillus anthracis, Bacillus subtilis, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, coagulase Negative Staphylococcus, Corynebacterium diphtheria, Enterococcus faecalis, Enterococcus faecium, Escherichia coli, enterotoxigenic E. coli, enteropathogenic E. coli, E. coli O157:H7, Enterobacter sp., Francisella tularensis, Haemophilus influenzae, Helicobacter pylori, Klebsiella pneumoniae, Legionella pneumophila, Leptospira interrogans, Listeria monocytogenes, Moraxella catarralis, Mycobacterium leprae, Mycobacterium tuberculosis, Mycoplasma pneumoniae, Neisseria gonorrhoeae, Neisseria meningitides, Preteus mirabilis, Proteus sps., Pseudomonas aeruginosa, Rickettsia rickettsii, Salmonella typhi, Salmonella typhimurium, Serratia marcesens, Shigella flexneri, Shigella sonnei, Staphylococcus aureus, Staphylococcus epidermidis, Staphylococcus saprophyticus, Streptococcus agalactiae, Streptococcus mutans, Streptococcus pneumoniae, Streptococcus pyogenes, Treponema pallidum, Vibrio cholerae, and Yersinia pestis.

In some embodiments, the pathogen is a fungal pathogen. Examples of fungal pathogens include, but are not limited to, Ascomycota (e.g., Fusarium oxysporum, Pneumocystis jirovecii, Aspergillus spp., Coccidioides immitis/posadasii, Candida albicans), Basidiomycota (e.g., Filobasidiella neoformans, Trichosporon), Microsporidia (e.g., Encephalitozoon cuniculi, Enterocytozoon bieneusi), and Mucoromycotina (e.g., Mucor circinelloides, Rhizopus oryzae, Lichtheimia corymbifera).

In some embodiments, the pathogen is a protozoan pathogen. Examples of protozoan pathogens include, but are not limited to, Entamoeba histolytica, Giardia lambila, Trichomonas vaginalis, Trypanosoma brucei, T. cruzi, Leishmania donovani, Balantidium coli, Toxoplasma gondii, Plasmodium spp., and Babesia microti.

In some embodiments, the pathogen is a parasitic pathogen. Examples of parasitic pathogens include, but are not limited to, Acanthamoeba, Anisakis, Ascaris lumbricoides, botfly, Balantidium coli, bedbug, Cestoda, chiggers, Cochliomyia hominivorax, Entamoeba histolytica, Fasciola hepatica, Giardia lamblia, hookworm, Leishmania, Linguatula serrata, liver fluke, Loa loa, Paragonimus, pinworm, Plasmodium falciparum, Schistosoma, Strongyloides stercoralis, mite, tapeworm, Toxoplasma gondii, Trypanosoma, whipworm, and Wuchereria bancrofti.

In some embodiments, the pathogen is an animal pathogen. Examples of animal pathogens, include, but are not limited to, bovine rhinotracheitis virus, bovine herpesvirus, distemper, parainfluenza (e.g., canine parainfluenza), canine adenovirus, rhinotracheitis virus, calicivirus, canine parvovirus, Borrelia burgdorferi (Lyme disease), Bordetella bronchiseptica (kennel cough), leptospirosis, feline immunodeficiency virus, feline leukemia virus, Dirofilaria immitis (heartworm), feline herpesvirus, Chlamydia infections, Bordetella infections, equine influenza, rhinopneumonitis (equine herpesvirus), equine encephalomyelitis, West Nile virus (equine), Streptococcus equi, tetanus (Clostridium tetani), equine protozoal myeloencephalitis, bovine respiratory disease complex, clostridial disease, bovine respiratory syncytial virus, bovine viral diarrhea, Haemophilus somnus, Pasteurella haemolytica, and Pastuerella multocida.

In some embodiments, the first target nucleic acid is a nucleic acid of a cancer cell. In some instances, for example, the first target nucleic acid encodes a tumor-associated antigen (TAA) and/or a neoantigen. Examples of TAAs include, but are not limited to, MelanA (MART-I), gplOO (Pmel 17), tyrosinase, TRP-I, TRP-2, MAGE-I, MAGE-3, BAGE, GAGE-I, GAGE-2, p15(58), CEA, RAGE, NY-ESO (LAGE), SCP-I, Hom/Mel-40, PRAME, p53, H-Ras, HER-2/neu, BCR-ABL, E2A-PRL, H4-RET, IGH-IGK, MYL-RAR, Epstein-Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, 3-HCG, BCA225, BTAA, CA 125, CA 15-3 (CA 27.29BCAA), CA 195, CA 242, CA-50, CAM43, CD68VKP1, CO-029, FGF-5, G250, Ga733 (EpCAM), HTgp-175, M344, MA-50, MG7-Ag, MOV18, NB/70K, NY-CO-I, RCAS1, SDCCAG16, TA-90 (Mac-2 binding proteincyclophilin C-associated protein), TAAL6, TAG72, TLP, and TPS5. Neoantigens, in some embodiments, arise from tumor proteins (e.g., tumor-associated antigens). In some embodiments, a neoantigen comprises a polypeptide comprising a sequence of approximately 10 to 250 amino acids that is identical to a sequence of amino acids within a tumor-associated antigen or an oncoprotein (e.g., Her2, E7, tyrosinase-related protein 2 (Trp2), Myc, Ras, or vascular endothelial growth factor (VEGF)).

In some embodiments, the first target nucleic acid is a nucleic acid of one or more contaminants (e.g., bacteria) of water, food, and/or soil.

In some embodiments, the first target nucleic acid is a nucleic acid associated with a single-nucleotide polymorphism (SNP). In certain cases, a diagnostic device may be used for rapid genotyping to detect the presence or absence of a SNP, which may affect medical treatment.

In some embodiments, a diagnostic device is configured to detect two or more target nucleic acids. In certain instances, the diagnostic device is configured to detect a nucleic acid of SARS-CoV-2 or a variant thereof and an influenza virus (e.g., an influenza A virus and/or an influenza B virus). In some embodiments, the diagnostic device is configured to detect at least 2 target nucleic acids, at least 3 target nucleic acids, at least 4 target nucleic acids, at least 5 target nucleic acids, at least 6 target nucleic acids, at least 7 target nucleic acids, at least 8 target nucleic acids, at least 9 target nucleic acids, or at least 10 target nucleic acids. In some embodiments, the diagnostic device is configured to detect 1 to 2 target nucleic acids, 1 to 5 target nucleic acids, 1 to 8 target nucleic acids, 1 to 10 target nucleic acids, 2 to 5 target nucleic acids, 2 to 8 target nucleic acids, 2 to 10 target nucleic acids, 5 to 8 target nucleic acids, 5 to 10 target nucleic acids, or 8 to 10 target nucleic acids. Each target nucleic acid may independently be a nucleic acid of a pathogen (e.g., a viral, bacterial, fungal, protozoan, or parasitic pathogen) and/or a cancer cell. In some embodiments, a diagnostic device has a relatively small size. In certain instances, the diagnostic device may be approximately the size of a pen or a marker. In some cases, the small size of the diagnostic device may advantageously allow the diagnostic device to be easily transported and/or easily stored in a home or business.

In some embodiments, the diagnostic device has a relatively short length (i.e., the longest dimension of the diagnostic device). In certain embodiments, the diagnostic device has a length of 30 cm or less, 25 cm or less, 20 cm or less, 15 cm or less, 10 cm or less, or 5 cm or less. In certain embodiments, the diagnostic device has a length in a range from 5 cm to 10 cm, 5 cm to 15 cm, 5 cm to 20 cm, 5 cm to 25 cm, 5 cm to 30 cm, 10 cm to 15 cm, 10 cm to 20 cm, 10 cm to 25 cm, 10 cm to 30 cm, 15 cm to 20 cm, 15 cm to 25 cm, 15 cm to 30 cm, 20 cm to 25 cm, 20 cm to 30 cm, or 25 cm to 30 cm. The length of the diagnostic device generally refers to the longest dimension of the diagnostic device in its initial configuration (e.g., prior to an inner component being pushed into an outer component).

In some embodiments, the diagnostic device has a relatively small maximum diameter (e.g., largest cross-sectional dimension). In some cases, a relatively small maximum diameter may advantageously facilitate use (e.g., by allowing users easily grasp the diagnostic device). In some embodiments, the diagnostic device has a maximum diameter of 5 cm or less, 4 cm or less, 3 cm or less, 2 cm or less, 1 cm or less, or 0.5 cm or less. In certain embodiments, the diagnostic device has a maximum diameter in a range from 0.5 cm to 1 cm, 0.5 cm to 2 cm, 0.5 cm to 3 cm, 0.5 cm to 4 cm, 0.5 cm to 5 cm, 1 cm to 2 cm, 1 cm to 3 cm, 1 cm to 4 cm, 1 cm to 5 cm, 2 cm to 3 cm, 2 cm to 4 cm, 2 cm to 5 cm, 3 cm to 4 cm, 3 cm to 5 cm, or 4 cm to 5 cm. The maximum diameter of the diagnostic device generally refers to the largest cross-sectional dimension of the diagnostic device, regardless of cross-sectional shape. The cross sections of the diagnostic device may have any suitable shape. For example, in certain embodiments, one or more cross sections of the diagnostic device may be substantially circular, substantially elliptical, substantially square, substantially rectangular, substantially triangular, or substantially irregular. In some embodiments, two or more cross sections of the diagnostic device may have substantially different shapes.

### Outer Component

In some embodiments, a diagnostic device comprises an outer component. The outer component may be associated with an inner component such that the inner component is movable relative to the outer component. In certain embodiments, at least a portion of the inner component may be pushed into at least a portion of the outer component. In certain embodiments, at least a portion of the inner component may be rotated relative to the outer component.

In some embodiments, the outer component comprises an outer casing. The outer casing may be formed from any suitable material. In some embodiments, the outer casing comprises a thermoplastic polymer and/or a metal. In certain embodiments, the outer casing is at least partially formed by injection molding. In certain embodiments, the outer casing is at least partially formed by an additive manufacturing process (e.g., 3D printing). In certain embodiments, the outer casing is at least partially formed by a subtractive manufacturing process (e.g., laser cutting). In certain embodiments, the outer casing comprises an opening through which at least a portion of the inner component and/or substrate are visible.

In some embodiments, the outer component may be configured to be secured to another component of a diagnostic kit (e.g., a reaction tube, a heating unit). In some cases, the outer component may be configured to be secured to another component of a diagnostic kit (e.g., a reaction tube, a heating unit) by a screw, a snap locking mechanism, and/or other fastener(s).

The outer component may have any suitable dimensions. In some embodiments, the outer component has a maximum diameter (e.g., largest cross-sectional dimension) of at least 0.5 cm, at least 1 cm, at least 2 cm, at least 3 cm, at least 4 cm, or at least 5 cm. In some embodiments, the outer component has a maximum diameter of 5 cm or less, 4 cm or less, 3 cm or less, 2 cm or less, 1 cm or less, or 0.5 cm or less. In certain embodiments, the outer component has a maximum diameter in a range from 0.5 cm to 1 cm, 0.5 cm to 2 cm, 0.5 cm to 3 cm, 0.5 cm to 4 cm, 0.5 cm to 5 cm, 1 cm to 2 cm, 1 cm to 3 cm, 1 cm to 4 cm, 1 cm to 5 cm, 2 cm to 3 cm, 2 cm to 4 cm, 2 cm to 5 cm, 3 cm to 4 cm, 3 cm to 5 cm, or 4 cm to 5 cm.

In some embodiments, the outer component has a length (e.g., longest dimension) of 25 cm or less, 20 cm or less, 15 cm or less, 10 cm or less, or 5 cm or less. In certain embodiments, the outer component has a length in a range from 5 cm to 10 cm, 5 cm to 15 cm, 5 cm to 20 cm, 5 cm to 25 cm, 10 cm to 15 cm, 10 cm to 20 cm, 10 cm to 25 cm, 15 cm to 20 cm, 15 cm to 25 cm, or 20 cm to 25 cm.

### Inner Component

In some embodiments, a diagnostic device comprises an inner component. In some embodiments, the inner component may be associated with a substrate. In certain cases, for example, the inner component may at least partially enclose at least a portion of a substrate. In some instances, a substrate may be removably coupled to the inner component. In some instances, a substrate may be permanently attached to (e.g., integrally formed with) the inner component. In certain embodiments, the inner component comprises an opening through which at least a portion of a substrate is visible.

The inner component may be formed from any suitable material. In some embodiments, the inner component comprises a thermoplastic polymer and/or a metal. In certain embodiments, the inner component is at least partially formed by injection molding. In certain embodiments, the inner component is at least partially formed by an additive manufacturing process (e.g., 3D printing). In certain embodiments, the inner component is at least partially formed by a subtractive manufacturing process (e.g., laser cutting).

The inner component may have any suitable dimensions. In some embodiments, at least a portion of the inner component has a diameter (e.g., largest cross-sectional dimension) that is less than a maximum diameter of the outer component. In certain embodiments, at least a portion of the inner component has a diameter of 4 cm or less, 3 cm or less, 2 cm or less, 1 cm or less, or 0.5 cm or less. In certain embodiments, at least a portion of the inner component has a diameter in a range from 0.5 cm to 1 cm, 0.5 cm to 2 cm, 0.5 cm to 3 cm, 0.5 cm to 4 cm, 1 cm to 2 cm, 1 cm to 3 cm, 1 cm to 4 cm, 2 cm to 3 cm, 2 cm to 4 cm, or 3 cm to 4 cm.

In some embodiments, the inner component has a maximum diameter that is less than a maximum diameter of the outer component. In certain embodiments, the maximum diameter of the inner component is 4 cm or less, 3 cm or less, 2 cm or less, 1 cm or less, or 0.5 cm or less. In certain embodiments, the maximum diameter of the inner component is in a range from 0.5 cm to 1 cm, 0.5 cm to 2 cm, 0.5 cm to 3 cm, 0.5 cm to 4 cm, 1 cm to 2 cm, 1 cm to 3 cm, 1 cm to 4 cm, 2 cm to 3 cm, 2 cm to 4 cm, or 3 cm to 4 cm.

In certain embodiments, the inner component has a relatively short length (e.g., the longest dimension of the inner component). In some embodiments, the inner component has a length of 25 cm or less, 20 cm or less, 15 cm or less, 10 cm or less, 5 cm or less, or 2 cm or less. In some embodiments, the inner component has a length in a range from 2 cm to 5 cm, 2 cm to 10 cm, 2 cm to 15 cm, 2 cm to 20 cm, 2 cm to 25 cm, 5 cm to 10 cm, 5 cm to 15 cm, 5 cm to 20 cm, 5 cm to 25 cm, 10 cm to 15 cm, 10 cm to 20 cm, 10 cm to 25 cm, 15 cm to 20 cm, or 15 cm to 25 cm.

### Sample-Collecting Component

In some embodiments, a diagnostic device comprises a sample-collecting component configured to collect a sample (e.g., a nasal secretion, an oral secretion, a genital secretion, a cell scraping, blood, urine) from a subject (e.g., a human subject, an animal subject). The sample-collecting component may be removably or permanently coupled to either an outer component or an inner component of a diagnostic device. In some embodiments, the sample-collecting component and either an outer component or an inner component of the diagnostic device form a unitary component.

In some embodiments, the sample-collecting component comprises a swab element. In certain instances, the swab element comprises an absorbent material. Examples of suitable absorbent materials include, but are not limited to, cotton, polyester, polyurethane, rayon, nylon, microfiber, viscose, cellulose, and alginate. In some instances, the swab element is a foam swab or a flocked swab (e.g., comprising flocked fibers of a material). In some instances, the swab element comprises a thermoplastic polymer and/or a metal. In some such instances, the swab element is formed by injection molding, an additive manufacturing process (e.g., 3D printing), and/or a subtractive manufacturing process (e.g., laser cutting).

The swab element may have any suitable shape and dimensions. In some embodiments, the swab element has a substantially conical shape. In certain embodiments, at least a portion of the swab element has a sufficiently small diameter (i.e., largest cross-sectional dimension) to facilitate insertion of the swab element into a reaction tube. In some embodiments, at least a portion of the swab element has a diameter less than a diameter of an opening of a reaction tube (e.g., a reaction tube of a diagnostic kit). In some embodiments, at least a portion of the swab element has a diameter less than a diameter of an opening of a heating unit (e.g., a heating unit of a diagnostic kit). In some embodiments, at least a portion of the swab element has a sufficiently small diameter to facilitate insertion of the swab element into a cavity of a subject. In some embodiments, at least a portion of the swab element has a sufficiently large diameter to allow at least a portion of the substrate (e.g., the reagent delivery region of the substrate) to move within at least a portion of the swab element.

In some embodiments, at least a portion of the swab element has a diameter of 20 mm or less, 15 mm or less, 10 mm or less, 9 mm or less, 8 mm or less, 7 mm or less, 6 mm or less, 5 mm or less, 4 mm or less, 3 mm or less, 2 mm or less, or 1 mm or less. In some embodiments, at least a portion of the swab element has a diameter of at least 1 mm, at least 2 mm, at least 3 mm, at least 4 mm, at least 5 mm, at least 6 mm, at least 7 mm, at least 8 mm, at least 9 mm, at least 10 mm, at least 15 mm, or at least 20 mm. In some embodiments, at least a portion of the swab element has a diameter in a range from 1 mm to 2 mm, 1 mm to 5 mm, 1 mm to 10 mm, 2 mm to 5 mm, 2 mm to 10 mm, 2 mm to 15 mm, 2 mm to 20 mm, 5 mm to 10 mm, 5 mm to 15 mm, 5 mm to 20 mm, 10 mm to 15 mm, or 10 mm to 20 mm.

In some embodiments, the swab element has a maximum diameter of 20 mm or less, 15 mm or less, 10 mm or less, 9 mm or less, 8 mm or less, 7 mm or less, 6 mm or less, 5 mm or less, 4 mm or less, 3 mm or less, 2 mm or less, or 1 mm or less. In some embodiments, the swab element has a maximum diameter of at least 1 mm, at least 2 mm, at least 3 mm, at least 4 mm, at least 5 mm, at least 6 mm, at least 7 mm, at least 8 mm, at least 9 mm, at least 10 mm, at least 15 mm, or at least 20 mm. In some embodiments, the swab element has a maximum diameter in a range from 1 mm to 2 mm, 1 mm to 5 mm, 1 mm to 10 mm, 2 mm to 5 mm, 2 mm to 10 mm, 2 mm to 15 mm, 2 mm to 20 mm, 5 mm to 10 mm, 5 mm to 15 mm, 5 mm to 20 mm, 10 mm to 15 mm, or 10 mm to 20 mm.

In some embodiments, the swab element has a relatively short length. In some instances, the swab element has a length of 20 mm or less, 15 mm or less, 10 mm or less, 9 mm or less, 8 mm or less, 7 mm or less, 6 mm or less, 5 mm or less, 4 mm or less, 3 mm or less, 2 mm or less, or 1 mm or less. In some instances, the swab element has a length in a range from 1 mm to 2 mm, 1 mm to 3 mm, 1 mm to 4 mm, 1 mm to 5 mm, 1 mm to 6 mm, 1 mm to 7 mm, 1 mm to 8 mm, 1 mm to 9 mm, 1 mm to 10 mm, 1 mm to 15 mm, 1 mm to 20 mm, 2 mm to 5 mm, 2 mm to 8 mm, 2 mm to 10 mm, 2 mm to 15 mm, 2 mm to 20 mm, 5 mm to 8 mm, 5 mm to 10 mm, 5 mm to 15 mm, 5 mm to 20 mm, 8 mm to 10 mm, 8 mm to 15 mm, 8 mm to 20 mm, 10 mm to 15 mm, 10 mm to 20 mm, or 15 mm to 20 mm.

In some embodiments, the length of the swab element is less than or equal to an initial depth of fluidic contents of the reaction tube (e.g., the depth of the fluidic contents of the reaction tube prior to insertion of the swab element). In certain instances, the length of the swab element is 100% or less, 95% or less, 90% or less, 80% or less, 70% or less, 60% or less, 50% or less, 40% or less, 30% or less, 20% or less, or 10% or less of the initial depth of fluidic contents of the reaction tube. In some embodiments, the length of the swab element is 10-20%, 10-50%, 10-80%, 10-90%, 10-95%, 10-100%, 20-50%, 20-80%, 20-90%, 20-95%, 20-100%, 50-80%, 50-90%, 50-95%, 50-100%, 80-90%, 80-95%, 80-100%, 90-100%, or 100% of the initial depth of fluidic contents of the reaction tube. In some instances, the swab element is at least partially submerged in the fluidic contents of the reaction tube (e.g., the one or more liquids of the reaction tube) after insertion into the reaction tube. In some instances, the swab element is fully submerged in the fluidic contents of the reaction tube (e.g., the one or more liquids of the reaction tube) after insertion into the reaction tube.

In some embodiments, the sample-collecting component comprises a stem element. The stem element may be proximal to the swab element of the sample-collecting component. In some embodiments, the stem element has a maximum diameter that is less than the maximum diameter of an outer casing of the outer component. In some embodiments, the relatively small maximum diameter of the stem element facilitates insertion of the sample-collecting component into a cavity (e.g., a nasal cavity, an oral cavity, a vaginal cavity, an anal cavity, an ear canal) of a subject (e.g., a human subject, an animal subject). In some embodiments, the stem element has a maximum diameter of 20 mm or less, 15 mm or less, 12 mm or less, 10 mm or less, 9 mm or less, 8 mm or less, 7 mm or less, 6 mm or less, 5 mm or less, 4 mm or less, 3 mm or less, 2 mm or less, or 1 mm or less. In some embodiments, the stem element has a diameter in a range from 1 mm to 5 mm, 1 mm to 10 mm, 1 mm to 12 mm, 1 mm to 15 mm, 1 mm to 20 mm, 5 mm to 10 mm, 5 mm to 12 mm, 5 mm to 15 mm, 5 mm to 20 mm, 10 mm to 15 mm, 10 mm to 20 mm, 12 mm to 20 mm, or 15 mm to 20 mm.

In some embodiments, the stem element has a length that is shorter than a length of the diagnostic device. In some embodiments, the stem element has a length of at least 1 cm, at least 2 cm, at least 3 cm, at least 4 cm, at least 5 cm, at least 6 cm, at least 7 cm, at least 8 cm, at least 9 cm, or at least 10 cm. In some embodiments, the stem element has a length of 10 cm or less, 9 cm or less, 8 cm or less, 7 cm or less, 6 cm or less, 5 cm or less, 4 cm or less, 3 cm or less, 2 cm or less, or 1 cm or less. In some embodiments, the stem element has a length in a range from 1 cm to 2 cm, 1 cm to 3 cm, 1 cm to 4 cm, 1 cm to 5 cm, 1 cm to 6 cm, 1 cm to 7 cm, 1 cm to 8 cm, 1 cm to 9 cm, 1 cm to 10 cm, 2 cm to 5 cm, 2 cm to 8 cm, 2 cm to 10 cm, 5 cm to 8 cm, 5 cm to 10 cm, or 8 cm to 10 cm.

### Substrate

Some embodiments are directed to a substrate. In some embodiments, the substrate comprises, in order of fluid flow direction, a reagent delivery region and a lateral flow assay region. In certain embodiments, the reagent delivery region comprises one or more reagents. In certain embodiments, the lateral flow assay region is configured to detect one or more target nucleic acids. In some instances, a separation region is positioned between the reagent delivery region and the lateral flow assay region. In some instances, the substrate is a single monolithic substrate. In some instances, the substrate comprises a plurality of discrete units (e.g., a series of separate lateral flow strips that are connected to facilitate fluid flow).

The substrate may have any suitable dimensions. In some embodiments, the substrate has a relatively short length (i.e., the longest dimension of the substrate). In certain embodiments, the substrate has a length of 25 cm or less, 20 cm or less, 15 cm or less, 10 cm or less, 5 cm or less, or 2 cm or less. In certain embodiments, the substrate has a length in a range from 2 cm to 5 cm, 2 cm to 10 cm, 2 cm to 15 cm, 2 cm to 20 cm, 2 cm to 25 cm, 5 cm to 10 cm, 5 cm to 15 cm, 5 cm to 20 cm, 5 cm to 25 cm, 10 cm to 15 cm, 10 cm to 20 cm, 10 cm to 25 cm, 15 cm to 20 cm, 15 cm to 25 cm, or 20 cm to 25 cm.

In some embodiments, the substrate has a relatively narrow maximum width. In certain embodiments, the substrate has a maximum width of 20 mm or less, 15 mm or less, 12 mm or less, 10 mm or less, 9 mm or less, 8 mm or less, 7 mm or less, 6 mm or less, 5 mm or less, 4 mm or less, 3 mm or less, 2 mm or less, or 1 mm or less. In some embodiments, the substrate has a maximum width in a range from 1 mm to 5 mm, 1 mm to 10 mm, 1 mm to 12 mm, 1 mm to 15 mm, 1 mm to 20 mm, 5 mm to 10 mm, 5 mm to 12 mm, 5 mm to 15 mm, 5 mm to 20 mm, 10 mm to 15 mm, 10 mm to 20 mm, or 15 mm to 20 mm.

In some embodiments, the substrate is relatively thin. In certain embodiments, the substrate has a maximum thickness of 5 mm or less, 4 mm or less, 3 mm or less, 2 mm or less, 1 mm or less, 0.9 mm or less, 0.8 mm or less, 0.7 mm or less, 0.6 mm or less, 0.5 mm or less, 0.4 mm or less, 0.3 mm or less, 0.2 mm or less, or 0.1 mm or less. In some embodiments, the substrate has a maximum thickness in a range from 0.1 mm to 0.2 mm, 0.1 mm to 0.3 mm, 0.1 mm to 0.4 mm, 0.1 mm to 0.5 mm, 0.1 mm to 0.6 mm, 0.1 mm to 0.7 mm, 0.1 mm to 0.8 mm, 0.1 mm to 0.9 mm, 0.1 mm to 1 mm, 0.1 mm to 2 mm, 0.1 mm to 5 mm, 0.2 mm to 0.4 mm, 0.2 mm to 0.5 mm, 0.2 mm to 0.6 mm, 0.2 mm to 0.7 mm, 0.2 mm to 0.8 mm, 0.2 mm to 0.9 mm, 0.2 mm to 1 mm, 0.2 mm to 2 mm, 0.2 mm to 5 mm, 0.5 mm to 1 mm, 0.5 mm to 2 mm, 0.5 mm to 5 mm, 1 mm to 2 mm, or 1 mm to 5 mm.

In some embodiments, the substrate comprises a base layer. In certain embodiments, the base layer comprises one or more materials that do not allow fluid transport (e.g., via capillary action). In some cases, the one or more materials of the base layer are substantially non-porous. Examples of suitable materials for the base layer include, but are not limited to, polymers (e.g., polyethylene terephthalate, polyethylene naphthalate, polyvinyl chloride, polyurethane), metals, metal alloys, and ceramics.

In some embodiments, the substrate comprises a reagent delivery region comprising one or more reagents. In some embodiments, the reagent delivery region comprises one or more fluid-transporting layers (e.g., positioned over the base layer of the substrate) comprising one or more materials that allow fluid transport (e.g., via capillary action). Non-limiting examples of suitable materials include polyethersulfone, cellulose, polycarbonate, nitrocellulose, sintered polyethylene, and glass fibers. In some embodiments, the one or more fluid-transporting layers comprise a plurality of fibers (e.g., woven or non-woven fabrics). In some embodiments, the one or more fluid-transporting layers comprise a plurality of pores. In some embodiments, pores and/or interstices between fibers may advantageously facilitate fluid transport (e.g., via capillary action).

In some embodiments, the one or more fluid-transporting layers comprise a plurality of pores. The pores may have any suitable average pore size. In certain embodiments, the plurality of pores has an average pore size of 30 μm or less, 25 μm or less, 20 μm or less, 15 μm or less, 10 μm or less, 5 μm or less, 2 μm or less, 1 μm or less, 0.9 μm or less, 0.8 μm or less, 0.7 μm or less, 0.6 μm or less, 0.5 μm or less, 0.4 μm or less, 0.3 μm or less, 0.2 μm or less, or 0.1 μm or less. In certain embodiments, the plurality of pores has an average pore size of at least 0.1 μm, at least 0.2 μm, at least 0.3 μm, at least 0.4 μm, at least 0.5 μm, at least 0.6 μm, at least 0.7 μm, at least 0.8 μm, at least 0.9 μm, at least 1 μm, at least 2 μm, at least 5 μm, at least 10 μm, at least 15 μm, at least 20 μm, at least 25 μm, or at least 30 μm. In some embodiments, the plurality of pores has an average pore size in a range from 0.1 μm to 0.5 μm, 0.1 μm to 1 μm, 0.1 μm to 5 μm, 0.1 μm to 10 μm, 0.1 μm to 15 μm, 0.1 μm to 20 μm, 0.1 μm to 25 μm, 0.1 μm to 30 μm, 0.5 μm to 1 μm, 0.5 μm to 5 μm, 0.5 μm to 10 μm, 0.5 μm to 15 μm, 0.5 μm to 20 μm, 0.5 μm to 25 μm, 0.5 μm to 30 μm, 1 μm to 5 μm, 1 μm to 10 μm, 1 μm to 15 μm, 1 μm to 20 μm, 1 μm to 25 μm, 1 μm to 30 μm, 5 μm to 10 μm, 5 μm to 15 μm, 5 μm to 20 μm, 5 μm to 25 μm, 5 μm to 30 μm, 10 μm to 15 μm, 10 μm to 20 μm, 10 μm to 25 μm, 10 μm to 30 μm, 15 μm to 20 μm, 15 μm to 25 μm, 15 μm to 30 μm, or 20 μm to 30 μm. Average pore size may be measured according to any method known in the art. For example, average pore size may be measured using scanning electron microscopy (SEM).

The one or more fluid-transporting layers may have any suitable porosity. In some embodiments, the one or more fluid-transporting layers have a porosity of at least 10%, at least 20%, at least 30%, at least 40%, at least 50%, or at least 60%. In some embodiments, the one or more fluid-transporting layers have a porosity in a range from 10% to 20%, 10% to 30%, 10% to 40%, 10% to 50%, 10% to 60%, 20% to 40%, 20% to 50%, 20% to 60%, 30% to 50%, 30% to 60%, 40% to 60%, or 50% to 60%. Porosity generally refers to the percentage of void volume of a material and may be measured according to any method known in the art. For example, porosity may be measured using SEM.

The reagent delivery region may have any suitable dimensions. In some embodiments, the reagent delivery region has a length of 10 mm or less, 9 mm or less, 8 mm or less, 7 mm or less, 6 mm or less, 5 mm or less, 4 mm or less, 3 mm or less, 2 mm or less, or 1 mm or less. The reagent delivery region may have a length in a range from 1 mm to 2 mm, 1 mm to 3 mm, 1 mm to 4 mm, 1 mm to 5 mm, 1 mm to 6 mm, 1 mm to 7 mm, 1 mm to 8 mm, 1 mm to 9 mm, 1 mm to 10 mm, 2 mm to 5 mm, 2 mm to 10 mm, 3 mm to 5 mm, 3 mm to 10 mm, 4 mm to 10 mm, 5 mm to 10 mm, 6 mm to 10 mm, 7 mm to 10 mm, 8 to 10 mm, or 9 to 10 mm.

In some embodiments, the reagent delivery region is configured to be inserted into a reaction tube, and the length of the reaction delivery region is less than an initial depth of fluidic contents of the reaction tube. In certain embodiments, the length of the reagent delivery region is 60% or less, 50% or less, 40% or less, 30% or less, 20% or less, 10% or less, 5% or less, or 1% or less of the initial depth of fluidic contents of the reaction tube. In some embodiments, the length of the reagent delivery region is 1-5%, 1-10%, 1-20%, 1-30%, 1-40%, 1-50%, 1-60%, 5-10%, 5-20%, 5-30%, 5-40%, 5-50%, 5-60%, 10-20%, 10-30%, 10-40%, 10-50%, 10-60%, 20-50%, 20-60%, 30-50%, 30-60%, 40-60%, or 50-60% of the initial depth of fluidic contents of the reaction tube. In some embodiments, the reagent delivery region is at least partially submerged in the fluidic contents of the reaction tube (e.g., the one or more liquids of the reaction tube) after insertion into the reaction tube (e.g., after one or more movements of the inner component relative to the outer component). In some embodiments, the reagent delivery region is fully submerged in the fluidic contents of the reaction tube (e.g., the one or more liquids of the reaction tube) after insertion into the reaction tube (e.g., after one or more movements of the inner component relative to the outer component).

In some embodiments, the reagent delivery region has a reagent delivery surface (e.g., a top surface) having an area of at least 0.1 cm², at least 0.2 cm², at least 0.3 cm², at least 0.4 cm², at least 0.5 cm², at least 0.6 cm², at least 0.7 cm², at least 0.8 cm², at least 0.9 cm², at least 1.0 cm², at least 1.2 cm², at least 1.5 cm², at least 1.8 cm², or at least 2.0 cm². In some embodiments, the reagent delivery surface has an area of 2.0 cm² or less, 1.8 cm² or less, 1.5 cm² or less, 1.2 cm² or less, 1.0 cm² or less, 0.9 cm² or less, 0.8 cm² or less, 0.7 cm² or less, 0.6 cm² or less, 0.5 cm² or less, 0.4 cm² or less, 0.3 cm² or less, 0.2 cm² or less, or 0.1 cm² or less. In some embodiments, the reagent delivery surface has an area in a range from 0.1 cm² to 0.5 cm², 0.1 cm² to 1.0 cm², 0.1 cm² to 1.5 cm², 0.1 cm² to 2.0 cm², 0.5 cm² to 1.0 cm², 0.5 cm² to 1.5 cm², 0.5 cm² to 2.0 cm², 1.0 cm² to 1.5 cm², 1.0 cm² to 2.0 cm², or 1.5 cm² to 2.0 cm².

The reagent delivery region may have any suitable shape. In some embodiments, the reagent delivery region has a reagent delivery surface (e.g., a top surface) that is substantially triangular, substantially rectangular, substantially trapezoidal, or any other suitable shape. In certain cases, a substantially triangular or trapezoidal shape may facilitate insertion of the reagent delivery region into a reaction tube.

In some embodiments, the reagent delivery region comprises one or more reagents. In some cases, at least one of the one or more reagents is thermostabilized (e.g., lyophilized, crystallized, air jetted, dried). In some cases, all of the one or more reagents are thermostabilized (e.g., lyophilized, crystallized, air jetted, dried). In certain embodiments, the one or more fluid-transporting layers may be impregnated with the one or more reagents.

In certain embodiments, the one or more reagents comprise one or more lysis reagents. A lysis reagent generally refers to a reagent that facilitates cell lysis. The lysis reagent may facilitate cell lysis alone or in combination with one or more additional reagents. In some embodiments, the one or more lysis reagents comprise one or more enzymes. Non-limiting examples of suitable enzymes include lysozyme, lysostaphin, zymolase, cellulase, protease, and glycanase. In some embodiments, the one or more lysis reagents comprise one or more detergents. Non-limiting examples of suitable detergents include sodium dodecyl sulphate (SDS), Tween (e.g., Tween 20, Tween 80), 3-[(3-cholamidopropyl)dimethylammonio]-1-propanesulfonate (CHAPS), 3-[(3-cholamidopropyl)dimethylammonio]-2-hydroxy-1-propanesulfonate (CHAPSO), Triton X-100, and NP-40.

In some embodiments, the one or more reagents comprise one or more reverse transcription reagents. A reverse transcription reagent generally refers to a reagent that facilitates reverse transcription of RNA to DNA (e.g., cDNA). In some embodiments, the one or more reverse transcription reagents comprise a reverse transcriptase, a DNA-dependent polymerase, and/or a ribonuclease (RNase). A reverse transcriptase generally refers to an enzyme that transcribes single-stranded RNA (ssRNA) into complementary DNA (cDNA) by polymerizing deoxyribonucleotide triphosphates (dNTPs). Examples of a suitable reverse transcriptase include, but are not limited to, HIV-1 reverse transcriptase, Moloney murine leukemia virus (M-MLV) reverse transcriptase, and avian myeloblastosis virus (AMV) reverse transcriptase. An RNase generally refers to an enzyme that catalyzes the degradation of RNA. In some cases, an RNase may be used to digest RNA from an RNA-DNA hybrid.

In some embodiments, the one or more reagents comprise one or more additives that enhance reagent stability (e.g., protein stability). Non-limiting examples of suitable additives include trehalose, polyethylene glycol (PEG), polyvinyl alcohol (PVA), and glycerol.

In some embodiments, the one or more reagents comprise one or more reagents to eliminate potential carryover contamination from prior tests conducted in the same area. In some embodiments, the one or more reagents comprise thermolabile uracil DNA glycosylase (UDG). In some cases, UDG may prevent carryover contamination from prior tests by degrading products that have already been amplified (i.e., amplicons) while leaving unamplified samples untouched and ready for amplification. In some embodiments, the concentration of UDG is at least 0.01 U/μL, at least 0.02 U/μL, at least 0.03 U/μL, at least 0.04 U/μL, or at least 0.05 U/μL. In certain embodiments, the concentration of UDG is in a range from 0.01 U/μL to 0.02 U/μL, 0.01 U/μL to 0.03 U/μL, 0.01 U/μL to 0.04 U/μL, or 0.01 U/μL to 0.05 U/μL.

In some embodiments, the one or more reagents comprise an RNase inhibitor (e.g., a murine RNase inhibitor). In certain embodiments, the RNase inhibitor concentration is at least 0.1 U/μL, at least 0.2 U/μL, at least 0.5 U/μL, at least 0.8 U/μL, at least 1.0 U/μL, at least 1.2 U/μL, at least 1.5 U/μL, at least 1.8 U/μL, or at least 2.0 U/μL. In certain embodiments, the RNase inhibitor concentration is in a range from 0.1 U/μL to 0.2 U/μL, 0.1 U/μL to 0.5 U/μL, 0.1 U/μL to 1.0 U/μL, 0.1 U/μL to 1.5 U/μL, 0.1 U/μL to 2.0 U/μL, 0.5 U/μL to 1.0 U/μL, 0.5 U/μL to 1.5 U/μL, 0.5 U/μL to 2.0 U/μL, or 1.0 U/μL to 2.0 U/μL.

In some embodiments, the one or more reagents comprise one or more nucleic acid amplification reagents. A nucleic acid amplification reagent generally refers to a reagent that facilitates a nucleic acid amplification method. In some embodiments, the nucleic acid amplification method is an isothermal nucleic acid amplification method. In some cases, an isothermal nucleic acid amplification method, unlike PCR, avoids use of expensive, bulky laboratory equipment for precise thermal cycling. Non-limiting examples of suitable isothermal nucleic acid amplification methods include loop-mediated isothermal amplification (LAMP), recombinase polymerase amplification (RPA), nicking enzyme amplification reaction (“NEAR”), thermophilic helicase dependent amplification (tHDA), nucleic acid sequence-based amplification (NASBA), strand displacement amplification (SDA), isothermal multiple displacement amplification (IMDA), rolling circle amplification (RCA), transcription mediated amplification (TMA), signal mediated amplification of RNA technology (SMART), single primer isothermal amplification (SPIA), circular helicase-dependent amplification (cHDA), and whole genome amplification (WGA).

In some embodiments, the nucleic acid amplification reagents are configured to amplify one or more nucleotide sequences of one or more target nucleic acids described herein (e.g., a nucleic acid of one or more pathogens).

In some embodiments, the nucleic acid amplification reagents are configured to amplify one or more nucleotide sequences of one or more control nucleic acids. A control nucleic acid is typically a gene or portion of a gene that is widely expressed and/or expressed at a high level in a control organism (e.g., a human or other mammal). In some cases, a control nucleic acid is a human or animal nucleic acid that is not associated with a pathogen, a cancer cell, or a contaminant. Examples of suitable control nucleic acids include, but are not limited to, RNase P, GAPDH, B2M, ACTB, POLR2A, UBC, PPIA, HPRT1, GUSB, TBP, H3F3A, POLR2A, RPLPO, L19, B2M, RPS17, ALAS1, CD74, CK18, HMBS, IPO8, PGK1, YWHAZ, and STATH. In some embodiments, successful amplification and detection of the control nucleic acid may indicate that a sample was properly collected and the diagnostic test was properly run. For example, successful amplification and detection of the control nucleic acid may indicate that the diagnostic test was properly run (e.g., sample was collected, cells were lysed, nucleic acids were amplified). On the other hand, failure to detect the control nucleic acid may indicate one or more of the following: improper specimen collection resulting in the lack of sufficient human sample material, improper extraction of nucleic acid from the sample, ineffective inhibition of RNAse in the sample, improper assay set up and execution, and reagent or equipment malfunction.

In some embodiments, the nucleic acid amplification reagents are LAMP reagents. LAMP refers to a method of amplifying a target nucleic acid using at least four primers through the creation of a series of stem-loop structures. Due to its use of multiple primers, LAMP may be highly specific for a target nucleic acid sequence.

In some embodiments, the LAMP reagents comprise four or more primers. In certain embodiments, the four or more primers comprise a forward inner primer (FIP), a backward inner primer (BIP), a forward outer primer (F3), and a backward outer primer (B3). In some cases, the four or more primers target at least six specific regions of a target gene. In some embodiments, the LAMP reagents further comprise a forward loop primer (Loop F or LF) and a backward loop primer (Loop B or LB). In certain cases, the loop primers target cyclic structures formed during amplification and can accelerate amplification.

Methods of designing LAMP primers are known in the art. In some cases, LAMP primers may be designed for each target nucleic acid a diagnostic device is configured to detect. For example, a diagnostic device configured to detect a first target nucleic acid (e.g., a nucleic acid of SARS-CoV-2 or a variant thereof) and a second target nucleic acid (e.g., a nucleic acid of an influenza virus) may comprise a first set of LAMP primers directed to the first target nucleic acid and a second set of LAMP primers directed to the second target nucleic acid. In some embodiments, the LAMP primers may be designed by alignment and identification of conserved sequences in a target pathogen (e.g., using Clustal X or a similar program) and then using a software program (e.g., PrimerExplorer). The specificity of different candidate primers may be confirmed using a BLAST search of the GenBank nucleotide database. Primers may be synthesized using any method known in the art.

In certain embodiments, the target pathogen is SARS-CoV-2 or a variant thereof. In some cases, primers for amplification of a SARS-CoV-2 nucleic acid sequence are selected from regions of the virus's nucleocapsid (N) gene, envelope (E) gene, membrane (M) gene, and/or spike (S) gene. In some instances, primers were selected from regions of the SARS-CoV-2 nucleocapsid (N) gene to maximize inclusivity across known SARS-CoV-2 strains and minimize cross-reactivity with related viruses and genomes that may be presence in the sample. Exemplary LAMP primers for detection of a SARS-CoV-2 nucleic acid sequence are provided in Table 1 below.

TABLE 1
Exemplary LAMP Primers (SARS-CoV-2)

| | | SEQ ID |
|---|---|---|
| Primer | Sequence (5′ to 3′) | NO: |
| F3_Set1 | CGGTGGACAAATTGTCAC | 1 |
| B3_Set1 | CTTCTCTGGATTTAACACACTT | 2 |
| Loop F_Set1 | TTACAAGCTTAAAGAATGTCTGAACACT | 3 |
| Loop B_Setl | TTGAATTTAGGTGAAACATTTGTCACG | 4 |
| FIP1_Set1 | TCAGCACACAAAGCCAAAAATTTATTTTTCTGTGCAAAG | 5 |
| | GAAATTAAGGAG | |
| BIP1_Set1 | TATTGGTGGAGCTAAACTTAAAGCCTTTTCTGTACAATC | 6 |
| | CCTTTGAGTG | |
| FIP2_Set1 | TCAGCACACAAAGCCAAAAATTTATCTGTGCAAAGGAA | 7 |
| | ATTAAGGAG | |
| BIP2_Set1 | TATTGGTGGAGCTAAACTTAAAGCCCTGTACAATCCCTT | 8 |
| | TGAGTG | |
| F3_Set2 | TGCTTCAGTCAGCTGATG | 9 |
| B3_Set2 | TTAAATTGTCATCTTCGTCCTT | 10 |
| FIP_Set2 | TCAGTACTAGTGCCTGTGCCCACAATCGTTTTTAAACGG | 11 |
| | GT | |
| BIP_Set2 | TCGTATACAGGGCTTTTGACATCTATCTTGGAAGCGACA | 12 |
| | ACAA | |
| Loop F_Set2 | CTGCACTTACACCGCAA | 13 |
| Loop B_Set2 | GTAGCTGGTTTTGCTAAATTCC | 14 |

In some embodiments, the LAMP reagents comprise a FIP and a BIP for one or more target nucleic acids. In some embodiments, the FTP and BIP each have a sequence that is at least 70%, at least 75%, at least 80%, at least 85%, at least 90%, at least 95%, at least 96%, at least 97%, at least 98%, at least 99%, at least 99.5%, or 100% identical to a primer sequence provided in Table 1 (e.g., SEQ ID NOS. 5 and 6, SEQ ID NOS. 7 and 8, SEQ ID NOS. 11 and 12). In some embodiments, the concentrations of FIP and BIP are each at least 0.5 μM, at least 0.6 μM, at least 0.7 μM, at least 0.8 μM, at least 0.9 μM, at least 1.0 μM, at least 1.1 μM, at least 1.2 μM, at least 1.3 μM, at least 1.4 μM, at least 1.5 μM, at least 1.6 μM, at least 1.7 μM, at least 1.8 μM, at least 1.9 μM, or at least 2.0 μM. In some embodiments, the concentrations of FIP and BIP are each in a range from 0.5 μM to 1 μM, 0.5 μM to 1.3 μM, 0.5 μM to 1.5 μM, 0.5 μM to 1.6 μM, 0.5 μM to 2.0 μM, 1 μM to 1.3 μM, 1 μM to 1.5 μM, 1 μM to 1.6 μM, 1 μM to 2 μM, or 1.5 μM to 2 μM.

In some embodiments, the LAMP reagents comprise an F3 primer and a B3 primer for one or more target nucleic acids. In some embodiments, the F3 primer and the B3 primer each have a sequence that is at least 70%, at least 75%, at least 80%, at least 85%, at least 90%, at least 95%, at least 96%, at least 97%, at least 98%, at least 99%, at least 99.5%, or 100% identical to a primer sequence provided in Table 1 (e.g., SEQ ID NOS. 1 and 2, SEQ ID NOS. 9 and 10). In some embodiments, the concentrations of the F3 primer and the B3 primer are each at least 0.05 μM, at least 0.1 μM, at least 0.15 μM, at least 0.2 μM, at least 0.25 μM, at least 0.3 μM, at least 0.35 μM, at least 0.4 μM, at least 0.45 μM, or at least 0.5 μM. In some embodiments, the concentrations of the F3 primer and the B3 primer are each in a range from 0.05 μM to 0.1 μM, 0.05 μM to 0.2 μM, 0.05 μM to 0.3 μM, 0.05 μM to 0.4 μM, 0.05 μM to 0.5 μM, 0.1 μM to 0.2 μM, 0.1 μM to 0.3 μM, 0.1 μM to 0.4 μM, 0.1 μM to 0.5 μM, 0.2 μM to 0.3 μM, 0.2 μM to 0.4 μM, 0.2 μM to 0.5 μM, 0.3 μM to 0.4 μM, 0.3 μM to 0.5 μM, or 0.4 μM to 0.5 μM.

In some embodiments, the LAMP reagents comprise a forward loop primer and a backward loop primer for one or more target nucleic acids. In some embodiments, the forward loop primer and the backward loop primer each have a sequence that is at least 70%, at least 75%, at least 80%, at least 85%, at least 90%, at least 95%, at least 96%, at least 97%, at least 98%, at least 99%, at least 99.5%, or 100% identical to a primer sequence provided in Table 1 (e.g., SEQ ID NOS. 3 and 4, SEQ ID NOS. 13 and 14). In some embodiments, the concentrations of the forward loop primer and the backward loop primer are each at least 0.1 μM, at least 0.2 μM, at least 0.3 μM, at least 0.4 μM, at least 0.5 μM, at least 0.6 μM, at least 0.7 μM, at least 0.8 μM, at least 0.9 μM, or at least 1.0 μM. In some embodiments, the concentrations of the forward loop primer and the backward loop primer are each in a range from 0.1 μM to 0.2 μM, 0.1 μM to 0.4 μM, 0.1 μM to 0.5 μM, 0.1 μM to 0.6 μM, 0.1 μM to 0.8 μM, 0.1 μM to 1.0 μM, 0.2 μM to 0.5 μM, 0.2 μM to 0.8 μM, 0.2 μM to 1.0 μM, 0.3 μM to 0.5 μM, 0.3 μM to 0.8 μM, 0.3 μM to 1.0 μM, 0.4 μM to 0.8 μM, 0.4 μM to 1.0 μM, 0.5 μM to 0.8 μM, 0.5 μM to 1.0 μM, or 0.8 μM to 1.0 μM.

In some embodiments, the LAMP reagents comprise LAMP primers designed to amplify one or more nucleotide sequences of one or more control nucleic acids. In some embodiments, the control nucleic acid is a nucleic acid sequence encoding human RNase P. Exemplary LAMP primers for RNase P are shown in Table 2. In some instances, the one or more LAMP reagents comprise at least four primers that each have a sequence that is at least 70%, at least 75%, at least 80%, at least 85%, at least 90%, at least 95%, at least 96%, at least 97%, at least 98%, at least 99%, at least 99.5%, or 100% identical to a primer sequence provided in Table 2.

TABLE 2
Exemplary RNase P Primers

| | | SEQ ID |
|---|---|---|
| Primer | Sequence (5′ to 3′) | NO: |
| F3 | TTGATGAGCTGGAGCCA | 15 |
| B3 | CACCCTCAATGCAGAGTC | 16 |
| FIP | GTGTGACCCTGAAGACTCGGTTTTAGCCACTGACTCGGA | 17 |
| | TC | |
| BIP | CCTCCGTGATATGGCTCTTCGTTTTTTTCTTACATGGCTC | 18 |
| | TGGTC | |
| Loop F | ATGTGGATGGCTGAGTTGTT | 19 |
| Loop B | CATGCTGAGTACTGGACCTC | 20 |
| Quencher | CAGCCATCCACAT-BHQ1 | 21 |

In some embodiments, one or more LAMP primers (e.g., one or more LAMP primers for one or more target nucleic acids, one or more LAMP primers for RNase P) comprise a label. Non-limiting examples of suitable labels include biotin, streptavidin, fluorescein isothiocyanate (FITC), fluorescein amidite (FAM), fluorescein, and digoxigenin (DIG). In some cases, labeling one or more LAMP primers may result in labeled amplicons, which may facilitate detection (e.g., via a lateral flow assay). In certain embodiments, the label is a fluorescent label. In some instances, the fluorescent label is associated with a quenching moiety that prevents the fluorescent label from signaling until the quenching moiety is removed. In certain embodiments, a LAMP primer is labeled with two or more labels.

In some embodiments, the LAMP reagents comprise a DNA polymerase with high strand displacement activity. Non-limiting examples of suitable DNA polymerases include a DNA polymerase long fragment (LF) of a thermophilic bacteria, such as Bacillus stearothermophilus (Bst), Bacillus Smithii (Bsm), Geobacillus sp. M (GspM), or Thermodesulfatator indicus (Tin), or a Taq DNA polymerase. In certain embodiments, the DNA polymerase is Bst LF DNA polymerase, GspM LF DNA polymerase, GspSSD LF DNA polymerase, Tin exo-LF DNA polymerase, or SD DNA polymerase. In each case, the DNA polymerase may be a wild type or mutant polymerase.

In some embodiments, the concentration of the DNA polymerase is at least 0.1 U/μL, at least 0.2 U/μL, at least 0.3 U/μL, at least 0.4 U/μL, at least 0.5 U/μL, at least 0.6 U/μL, at least 0.7 U/μL, at least 0.8 U/μL, at least 0.9 U/μL, or at least 1.0 U/μL. In some embodiments, the concentration of the DNA polymerase is in a range from 0.1 U/μL to 0.5 U/μL, 0.1 U/μL to 1.0 U/μL, 0.2 U/μL to 0.5 U/μL, 0.2 U/μL to 1.0 U/μL, or 0.5 U/μL to 1.0 U/μL.

In some embodiments, the LAMP reagents comprise deoxyribonucleotide triphosphates (“dNTPs”). In certain embodiments, the LAMP reagents comprise deoxyadenosine triphosphate (“dATP”), deoxyguanosine triphosphate (“dGTP”), deoxycytidine triphosphate (“dCTP”), and deoxythymidine triphosphate (“dTTP”). In certain embodiments, the concentration of each dNTP (i.e., dATP, dGTP, dCTP, dTTP) is at least 0.5 mM, at least 0.6 mM, at least 0.7 mM, at least 0.8 mM, at least 0.9 mM, at least 1.0 mM, at least 1.1 mM, at least 1.2 mM, at least 1.3 mM, at least 1.4 mM, at least 1.5 mM, at least 1.6 mM, at least 1.7 mM, at least 1.8 mM, at least 1.9 mM, or at least 2.0 mM. In some embodiments, the concentration of each dNTP is in a range from 0.5 mM to 1.0 mM, 0.5 mM to 1.5 mM, 0.5 mM to 2.0 mM, 1.0 mM to 1.5 mM, 1.0 mM to 2.0 mM, or 1.5 mM to 2.0 mM.

In some embodiments, the LAMP reagents comprise magnesium sulfate (MgSO₄). In certain embodiments, the concentration of MgSO₄ is at least 1 mM, at least 2 mM, at least 3 mM, at least 4 mM, at least 5 mM, at least 6 mM, at least 7 mM, at least 8 mM, at least 9 mM, or at least 10 mM. In certain embodiments, the concentration of MgSO₄ is in a range from 1 mM to 2 mM, 1 mM to 5 mM, 1 mM to 8 mM, 1 mM to 10 mM, 2 mM to 5 mM, 2 mM to 8 mM, 2 mM to 10 mM, 5 mM to 8 mM, 5 mM to 10 mM, or 8 mM to 10 mM.

In some embodiments, the LAMP reagents comprise betaine. In certain embodiments, the concentration of betaine is at least 0.1 M, at least 0.2 M, at least 0.3 M, at least 0.4 M, at least 0.5 M, at least 0.6 M, at least 0.7 M, at least 0.8 M, at least 0.9 M, at least 1.0 M, at least 1.1 M, at least 1.2 M, at least 1.3 M, at least 1.4 M, or at least 1.5 M. In certain embodiments, the concentration of betaine is in a range from 0.1 M to 0.2 M, 0.1 M to 0.5 M, 0.1 M to 0.8 M, 0.1 M to 1.0 M, 0.1 M to 1.2 M, 0.1 M to 1.5 M, 0.2 M to 0.5 M, 0.2 M to 0.8 M, 0.2 M to 1.0 M, 0.2 M to 1.2 M, 0.2 M to 1.5 M, 0.5 M to 0.8 M, 0.5 M to 1.0 M, 0.5 M to 1.2 M, 0.5 M to 1.5 M, 0.8 M to 1.0 M, 0.8 M to 1.2 M, 0.8 M to 1.5 M, 1.0 M to 1.2 M, or 1.0 M to 1.5 M.

In some embodiments, the nucleic acid amplification reagents are RPA reagents. RPA generally refers to a method of amplifying a target nucleic acid using a recombinase, a single-stranded DNA binding protein, and a strand-displacing polymerase.

In some embodiments, the RPA reagents comprise a probe, a forward primer, and a reverse primer. The probe, forward primer, and reverse primer may be designed for each target nucleic acid a diagnostic device is configured to detect. In certain embodiments, each primer comprises at least 15 base pairs, at least 20 base pairs, at least 25 base pairs, at least 30 base pairs, at least 35 base pairs, at least 40 base pairs, at least 45 base pairs, or at least 50 base pairs. In certain embodiments, each primer comprises 15-20 base pairs, 15-30 base pairs, 15-40 base pairs, 15-50 base pairs, 20-30 base pairs, 20-40 base pairs, 20-50 base pairs, 30-40 base pairs, 30-50 base pairs, or 40-50 base pairs. In some embodiments, each primer does not have any mismatches within 3 base pairs of its 3′ terminus. In some embodiments, each primer comprises 10 or fewer, 9 or fewer, 8 or fewer, 7 or fewer, 6 or fewer, 5 or fewer, 4 or fewer, 3 or fewer, 2 or fewer, 1 or fewer, or no mismatches. In some embodiments, each mismatch is at least 3 base pairs, at least 4 base pairs, at least 5 base pairs, at least 6 base pairs, at least 7 base pairs, at least 8 base pairs, at least 9 base pairs, or at least 10 base pairs from the 3′ terminus. While mismatches more than 3 base pairs away from the 3′ terminus of the primer have been found to be well tolerated in RPA, multiple mismatches within 3 base pairs of the 3′ terminus have been found to inhibit the reaction.

As an illustrative example, in some instances, a first target nucleic acid is a nucleic acid of SARS-CoV-2. Exemplary RPA primers for detection of a nucleic acid sequence from the SARS-CoV-2 nucleocapsid (N) gene are provided in Table 3 below.

TABLE 3
Exemplary Recombination Polymerase Amplification Primers

| | | SEQ |
|---|---|---|
| | | ID |
| RPA_primer | Sequence | NOs: |
| Forward | GTACTGCCACTAAAGCATACAATGTAACAC | 22 |
| Primer | | |
| Reverse | {6-FAM}AATATGCTTATTCAGCAAAATGACTTGATCT | 23 |
| Primer | | |
| Probe | {biotin}CAGACAAGGAACTGATTACAAACATTGGCCGCA{d | 24, 25 |
| | Spacer}ATTGCACAATTTGCC{phos} | |

In some embodiments, the RPA reagents comprise a forward primer. In certain embodiments, the forward primer is at least 70%, at least 75%, at least 80%, at least 85%, at least 90%, at least 95%, at least 99%, or 100% identical to SEQ ID NO: 22. In some embodiments, the forward primer is at least 1 base pair, at least 2 base pairs, at least 3 base pairs, at least 4 base pairs, or at least 5 base pairs longer or shorter than SEQ ID NO: 22. In some embodiments, the forward primer comprises an antigenic tag. In certain embodiments, the concentration of the forward primer is at least 100 nM, at least 200 nM, at least 300 nM, at least 400 nM, at least 500 nM, at least 600 nM, at least 700 nM, at least 800 nM, at least 900 nM, or at least 1000 nM. In certain embodiments, the concentration of the forward primer is in a range from 100 nM to 200 nM, 100 nM to 500 nM, 100 nM to 800 nM, 100 nM to 1000 nM, 200 nM to 500 nM, 200 nM to 800 nM, 200 nM to 1000 nM, 500 nM to 800 nM, 500 nM to 1000 nM, or 800 nM to 1000 nM.

In some embodiments, the RPA reagents comprise a reverse primer. In certain embodiments, the reverse primer is at least 70%, at least 75%, at least 80%, at least 85%, at least 90%, at least 95%, at least 99%, or 100% identical to SEQ ID NO: 23. In some embodiments, the reverse primer is at least 1 base pair, at least 2 base pairs, at least 3 base pairs, at least 4 base pairs, or at least 5 base pairs longer or shorter than SEQ ID NO: 23. In some embodiments, the reverse primer comprises an antigenic tag. In certain embodiments, the concentration of the reverse primer is at least 100 nM, at least 200 nM, at least 300 nM, at least 400 nM, at least 500 nM, at least 600 nM, at least 700 nM, at least 800 nM, at least 900 nM, or at least 1000 nM. In certain embodiments, the concentration of the reverse primer is in a range from 100 nM to 200 nM, 100 nM to 500 nM, 100 nM to 800 nM, 100 nM to 1000 nM, 200 nM to 500 nM, 200 nM to 800 nM, 200 nM to 1000 nM, 500 nM to 800 nM, 500 nM to 1000 nM, or 800 nM to 1000 nM. In some embodiments, the RPA reagents further comprises a probe. In certain embodiments, the probe is at least 70%, at least 75%, at least 80%, at least 85%, at least 90%, at least 95%, at least 99%, or 100% identical to SEQ ID NOS: 24-25. In some embodiments, the concentration of the probe is at least 50 nM, at least 60 nM, at least 70 nM, at least 80 nM, at least 90 nM, at least 100 nM, at least 110 nM, at least 120 nM, at least 130 nM, at least 140 nM, at least 150 nM, at least 160 nM, at least 170 nM, at least 180 nM, at least 190 nM, or at least 200 nM. In some embodiments, the concentration of the probe is in a range from 50 nM to 100 nM, 50 nM to 120 nM, 50 nM to 150 nM, 50 nM to 180 nM, 50 nM to 200 nM, 100 nM to 120 nM, 100 nM to 150 nM, 100 nM to 180 nM, 100 nM to 200 nM, 120 nM to 180 nM, 120 nM to 200 nM, or 150 nM to 200 nM.

In some embodiments, the RPA reagents comprise RPA primers designed to amplify one or more nucleotide sequences of one or more control nucleic acids. In some embodiments, the control nucleic acid is a nucleic acid sequence encoding human RNase P. In some embodiments, the RPA reagents comprise primers (e.g., forward primers, reverse primers) and probes configured to detect a nucleic acid sequence encoding human RNase P.

In some embodiments, the RPA reagents comprise one or more recombinase enzymes. Non-limiting examples of suitable recombinase enzymes include T4 UvsX protein and T4 UvsY protein. In some embodiments, the concentration of each recombinase enzyme is at least 0.01 mg/mL, at least 0.02 mg/mL, at least 0.03 mg/mL, at least 0.04 mg/mL, at least 0.05 mg/mL, at least 0.06 mg/mL, at least 0.07 mg/mL, at least 0.08 mg/mL, at least 0.09 mg/mL, at least 0.10 mg/mL, at least 0.11 mg/mL, at least 0.12 mg/mL, at least 0.13 mg/mL, at least 0.14 mg/mL, or at least 0.15 mg/mL. In some embodiments, the concentration of each recombinase enzyme is in a range from 0.01 mg/mL to 0.05 mg/mL, 0.01 mg/mL to 0.1 mg/mL, 0.01 mg/mL to 0.15 mg/mL, 0.05 mg/mL to 0.1 mg/mL, 0.05 mg/mL to 0.15 mg/mL, or 0.10 mg/mL to 0.15 mg/mL.

In some embodiments, the RPA reagents comprise one or more single-stranded DNA binding proteins. A non-limiting example of a suitable single-stranded DNA binding protein is T4 gp32 protein. In certain embodiments, the concentration of the single-stranded DNA binding protein is at least 0.1 mg/mL, at least 0.2 mg/mL, at least 0.3 mg/mL, at least 0.4 mg/mL, at least 0.5 mg/mL, at least 0.6 mg/mL, at least 0.7 mg/mL, at least 0.8 mg/mL, at least 0.9 mg/mL, or at least 1.0 mg/mL. In certain embodiments, the concentration of the single-stranded DNA binding protein is in a range from 0.1 mg/mL to 0.2 mg/mL, 0.1 mg/mL to 0.5 mg/mL, 0.1 mg/mL to 0.8 mg/mL, 0.1 mg/mL to 1.0 mg/mL, 0.2 mg/mL to 0.5 mg/mL, 0.2 mg/mL to 0.8 mg/mL, 0.2 mg/mL to 1.0 mg/mL, 0.5 mg/mL to 0.8 mg/mL, 0.5 mg/mL to 1.0 mg/mL, or 0.8 mg/mL to 1.0 mg/mL.

In some embodiments, the RPA agents comprise a DNA polymerase. A non-limiting example of a suitable DNA polymerase is Staphylococcus aureus DNA polymerase (Sau). In certain embodiments, the concentration of the DNA polymerase is at least 0.01 mg/mL, at least 0.02 mg/mL, at least 0.03 mg/mL, at least 0.04 mg/mL, at least 0.05 mg/mL, at least 0.06 mg/mL, at least 0.07 mg/mL, at least 0.08 mg/mL, at least 0.09 mg/mL, or at least 0.1 mg/mL. In certain embodiments, the concentration of the single-stranded DNA binding protein is in a range from 0.01 mg/mL to 0.02 mg/mL, 0.01 mg/mL to 0.05 mg/mL, 0.01 mg/mL to 0.08 mg/mL, 0.01 mg/mL to 0.1 mg/mL, 0.02 mg/mL to 0.05 mg/mL, 0.02 mg/mL to 0.08 mg/mL, 0.02 mg/mL to 0.1 mg/mL, 0.05 mg/mL to 0.08 mg/mL, 0.05 mg/mL to 0.1 mg/mL, or 0.08 mg/mL to 0.1 mg/mL.

In some embodiments, the RPA agents comprise an endonuclease. A non-limiting example of a suitable endonuclease is Endonuclease IV. In some embodiments, the concentration of the endonuclease is at least 0.001 mg/mL, at least 0.002 mg/mL, at least 0.003 mg/mL, at least 0.004 mg/mL, at least 0.005 mg/mL, at least 0.006 mg/mL, at least 0.007 mg/mL, at least 0.008 mg/mL, at least 0.009 mg/mL, at least 0.01 mg/mL, at least 0.02 mg/mL, or at least 0.05 mg/mL. In some embodiments, the concentration of the endonuclease is in a range from 0.001 mg/mL to 0.005 mg/mL, 0.001 mg/mL to 0.01 mg/mL, 0.001 mg/mL to 0.02 mg/mL, 0.001 mg/mL to 0.05 mg/mL, 0.005 mg/mL to 0.01 mg/mL, 0.005 mg/mL to 0.02 mg/mL, 0.005 mg/mL to 0.05 mg/mL, 0.01 mg/mL to 0.02 mg/mL, or 0.01 mg/mL to 0.05 mg/mL.

In some embodiments, the RPA reagents comprise dNTPs (e.g., dATP, dGTP, dCTP, dTTP). In certain embodiments, the concentration of each dNTP is at least 0.1 mM, at least 0.2 mM, at least 0.3 mM, at least 0.4 mM, at least 0.5 mM, at least 0.6 mM, at least 0.7 mM, at least 0.8 mM, at least 0.9 mM, at least 1.0 mM, at least 1.1 mM, at least 1.2 mM, at least 1.3 mM, at least 1.4 mM, at least 1.5 mM, at least 1.6 mM, at least 1.7 mM, at least 1.8 mM, at least 1.9 mM, or at least 2.0 mM. In some embodiments, the concentration of each dNTP is in a range from 0.1 mM to 0.2 mM, 0.1 mM to 0.5 mM, 0.1 mM to 0.8 mM, 0.1 mM to 1.0 mM, 0.1 mM to 1.5 mM, 0.1 mM to 2.0 mM, 0.2 mM to 0.5 mM, 0.2 mM to 0.8 mM, 0.2 mM to 1.0 mM, 0.2 mM to 1.5 mM, 0.2 mM to 2.0 mM, 0.5 mM to 1.0 mM, 0.5 mM to 1.5 mM, 0.5 mM to 2.0 mM, 1.0 mM to 1.5 mM, 1.0 mM to 2.0 mM, or 1.5 mM to 2.0 mM.

In some embodiments, the RPA reagents comprise one or more additional components. Non-limiting examples of suitable components include DL-Dithiothreitol, phosphocreatine disodium hydrate, creatine kinase, and adenosine 5′-triphosphate disodium salt.

In some embodiments, the nucleic acid amplification reagents are NEAR reagents. NEAR generally refers to a method for amplifying a target nucleic acid using a nicking endonuclease and a strand displacing DNA polymerase. In some cases, NEAR may allow for amplification of very small amplicons.

In some embodiments, the NEAR reagents comprise a forward primer. In certain instances, the forward primer comprises a hybridization region at the 3′ end that is complementary to the 3′ end of a target gene antisense strand, a nicking enzyme binding site and a nicking site upstream of the hybridization region, and a stabilizing region upstream of the nicking site. In some embodiments, the NEAR reagents comprise a reverse primer. In certain instances, the reverse primer comprises a hybridization region at the 3′ end that is complementary to the 3′ end of a target gene sense strand, a nicking enzyme binding site and a nicking site upstream of the hybridization region, and a stabilizing region upstream of the nicking site. In some embodiments, the NEAR reagents comprise a probe. In certain embodiments, the probe comprises a complementary nucleic acid sequence to a target gene nucleic acid sequence. In some embodiments, the probe is conjugated to a detectable label. In some instances, the detectable label is a fluorophore, an enzyme, a quencher, an enzyme inhibitor, a radioactive label, a member of a binding pair, or a combination thereof.

In some embodiments, the NEAR reagents comprise a DNA polymerase. Examples of a suitable DNA polymerase include, but are not limited to, Geobacillus bogazici DNA polymerase, Bst (large fragment) DNA Polymerase, and Manta 1.0 DNA Polymerase (Enzymatics 3 e). In some embodiments, the NEAR reagents comprise one or more nicking enzymes. Non-limiting examples of suitable nicking enzymes include Nt. BspQI, Nb. BbvCi, Nb. BsmI, Nb. BsrDI, Nb. BtsI, Nt. AlwI, Nt. BbvCI, Nt. BstNBI, Nt. CviPII, Nb. Bpul OI, Nt. BpulOI, and N. BspD61. In some embodiments, the NEAR reagents comprise dNTPs (e.g., dATP, dGTP, dCTP, dTTP).

In some embodiments, the nucleic acid amplification reagents are tHDA reagents. In certain embodiments, the tHDA reagents comprise a helicase. A non-limiting example of a suitable helicase is UvrD helicase. In some embodiments, the tHDA reagents comprise a DNA polymerase. Non-limiting examples of suitable DNA polymerases include Bst LF DNA polymerase, GspM LF DNA polymerase, GspSSD LF DNA polymerase, Tin exo-LF DNA polymerase, and SD DNA polymerase. In each case, the DNA polymerase may be a wild type or mutant polymerase. In some embodiments, the tHDA reagents comprise one or more restriction enzymes. Non-limiting examples of suitable restriction enzymes include MPoI restriction enzyme and Hpy 18811 restriction enzyme.

In some embodiments, the tHDA reagents comprise a forward primer and a reverse primer. In some embodiments, the tHDA reagents further comprise a probe. In certain cases, the forward primer, the reverse primer, and/or probe are labeled. Examples of suitable labels include, but are not limited to, biotin, streptavidin, fluorescein isothiocyanate (FITC), fluorescein amidite (FAM), fluorescein, and digoxigenin (DIG). In some embodiments, the tHDA reagents comprise one or more additional reagents. Non-limiting examples of suitable reagents include Ficoll 400, MgSO₄, and NaCl.

In some embodiments, the one or more reagents comprise one or more reagents for CRISPR/Cas detection. CRISPR generally refers to Clustered Regularly Interspaced Short Palindromic Repeats, and Cas generally refers to a particular family of proteins. In some cases, a CRISPR/Cas detection platform can be combined with an isothermal amplification method to create a single step reaction (Joung et al., “Point-of-care testing for COVID-19 using SHERLOCK diagnostics,” 2020). For example, the amplification and CRISPR detection methods may be performed using reagents having compatible chemistries (e.g., reagents that do not interact detrimentally with one another and are sufficiently active to perform amplification and detection). In some embodiments, CRISPR/Cas detection method is combined with LAMP.

CRISPR/Cas detection platforms are known in the art. Examples of such platforms include SHERLOCK® and DETECTR® (see, e.g., Kellner et al., Nature Protocols, 2019, 14: 2986-3012; Broughton et al., Nature Biotechnology, 2020; Joung et al., 2020). In some embodiments, CRISPR/Cas methods are used to detect a target nucleic acid sequence (e.g., from a pathogen). In particular, a guide nucleic acid designed to recognize a target nucleic acid sequence (e.g., a SARS-CoV-2-specific sequence) may be used to detect target nucleic acid sequences present in a sample. If the sample comprises the target nucleic acid sequence, gRNA will bind to the target nucleic acid sequence and activate a programmable nuclease (e.g., a Cas protein), which may then cleave a reporter molecule and release a detectable moiety (e.g., a reporter molecule tagged with specific antibodies, a fluorophore, a dye, a polypeptide). In some embodiments, the detectable moiety binds to a capture reagent (e.g., an antibody) on a lateral flow strip, as described herein.

In some embodiments, the one or more reagents for CRISPR/Cas detection comprise one or more guide nucleic acids. As noted above, a guide nucleic acid may comprise a segment with reverse complementarity to a segment of the target nucleic acid sequence. In some embodiments, the guide nucleic acid is selected from a group of guide nucleic acids that have been screened against the nucleic acid of a strain of an infection or genomic locus of interest. In certain instances, for example, the guide nucleic acid may be selected from a group of guide nucleic acids that have been screened against the nucleic acid of a strain of SARS-CoV-2. In some embodiments, guide nucleic acids that are screened against the nucleic acid of a target sequence of interest can be pooled. Without wishing to be bound by a particular theory, it is thought that pooled guide nucleic acids directed against a single target nucleic acid can ensure broad coverage of the target nucleic acid within a single reaction. The pooled guide nucleic acids, in some embodiments, are directed to different regions of the target nucleic acid and may be sequential or non-sequential.

In some embodiments, a guide nucleic acid comprises a crRNA and/or tracrRNA. The guide nucleic acid may not be naturally occurring and may be made by artificial combination of otherwise separate segments of sequence. For example, in some embodiments, an artificial guide nucleic acid may be synthesized by chemical synthesis, genetic engineering techniques, and/or artificial manipulation of isolated segments of nucleic acids. In some embodiments, the targeting region of a guide nucleic acid is at least 10, 15, 20, 25, 30, 35, 40, 45, 50, 55, or 60 nucleotides (nt) in length. In some embodiments, the targeting region of a guide nucleic acid has a length in a range from 10 to 20 nt, 10 to 30 nt, 10 to 40 nt, 10 to 50 nt, 10 to 60 nt, 20 to 30 nt, 20 to 40 nt, 20 to 50 nt, 20 to 60 nt, 30 to 40 nt, 30 to 50 nt, 30 to 60 nt, 40 to 50 nt, 40 to 60 nt, or 50 to 60 nt.

In some embodiments, the one or more reagents for CRISPR/Cas detection comprise one or more programmable nucleases. In some embodiments, a programmable nuclease is capable of sequence-independent cleavage after the gRNA binds to its specific target sequence. In some instances, the programmable nuclease is a Cas protein. Non-limiting examples of suitable Cas proteins include Cas9, Cas12a, Cas12b, Cas13, and Cas14. In general, Cas9 and Cas12 nucleases are DNA-specific, Cas13 is RNA-specific, and Cas14 targets single-stranded DNA.

In some embodiments, the one or more reagents for CRISPR/Cas detection comprise a plurality of guide nucleic acids and a plurality of programmable nucleases. In some embodiments, each guide nucleic acid of the plurality of guide nucleic acids targets a different nucleic acid and is associated with a different programmable nuclease. As an illustrative example, if a diagnostic device is configured to detect two different target nucleic acids, the one or more CRISPR/Cas reagents may comprise at least two different guide nucleic acids and at least two different programmable nucleases. If two target nucleic acids are present in a sample, then two different programmable nucleases will be activated, which will result in the release of two unique detectable moieties. Thus, in this manner, the CRISPR/Cas detection system may be used to detect more than one target nucleic acid. In some embodiments, the CRISPR/Cas detection system may be used to detect at least 2, at least 3, at least 4, at least 5, at least 6, at least 7, at least 8, at least 9, or at least 10 target nucleic acids.

In some embodiments, the substrate comprises a separation region positioned adjacent to the reagent delivery region. In certain embodiments, the separation region is positioned directly adjacent to the reaction delivery region. In some embodiments, the separation region comprises one or more materials that do not allow fluid transport (e.g., via capillary action). Moreover, in some embodiments, the separation region does not comprise any materials that allow fluid transport (e.g., via capillary action). In some cases, the one or more materials of the separation region are substantially non-porous. Examples of suitable materials for the separation region include, but are not limited to, polymers (e.g., polyethylene terephthalate, polyethylene naphthalate, polyvinyl chloride, polyurethane), metals, metal alloys, and ceramics.

The separation region may have any suitable dimensions. In some embodiments, the separation region has a length of 30 mm or less, 25 mm or less, 20 mm or less, 15 mm or less, 12 mm or less, 10 mm or less, 9 mm or less, 8 mm or less, 7 mm or less, 6 mm or less, 5 mm or less, 4 mm or less, 3 mm or less, 2 mm or less, or 1 mm or less. In some embodiments, the separation region has a length in a range from 1 mm to 5 mm, 1 mm to 10 mm, 1 mm to 15 mm, 1 mm to 20 mm, 1 mm to 25 mm, 1 mm to 30 mm, 2 mm to 5 mm, 2 mm to 10 mm, 2 mm to 15 mm, 2 mm to 20 mm, 2 mm to 25 mm, 2 mm to 30 mm, 5 mm to 10 mm, 5 mm to 15 mm, 5 mm to 20 mm, 5 mm to 25 mm, 5 mm to 30 mm, 10 mm to 15 mm, 10 mm to 20 mm, 10 mm to 25 mm, 10 mm to 30 mm, 15 mm to 20 mm, 15 mm to 25 mm, 15 mm to 30 mm, 20 mm to 25 mm, 20 mm to 30 mm, or 25 mm to 30 mm.

In some embodiments, the separation region is configured to be inserted into a reaction tube, and the length of the separation region is less than an initial depth of fluidic contents of the reaction tube. In certain embodiments, the length of the separation region is 60% or less, 50% or less, 40% or less, 30% or less, 20% or less, 10% or less, 5% or less, or 1% or less of the initial depth of fluidic contents of the reaction tube. In some embodiments, the length of the separation region is 1-5%, 1-10%, 1-20%, 1-30%, 1-40%, 1-50%, 1-60%, 5-10%, 5-20%, 5-30%, 5-40%, 5-50%, 5-60%, 10-20%, 10-30%, 10-40%, 10-50%, 10-60%, 20-50%, 20-60%, 30-50%, 30-60%, 40-60%, or 50-60% of the initial depth of fluidic contents of the reaction tube.

In some embodiments, the substrate comprises a lateral flow assay region. In some embodiments, the lateral flow assay region is configured to detect one or more target nucleic acids. In certain cases, the lateral flow assay region comprises one or more fluid-transporting layers (e.g., positioned over the base layer of the substrate) comprising one or more materials that allow fluid transport (e.g., via capillary action). Non-limiting examples of suitable materials include polyethersulfone, cellulose, polycarbonate, nitrocellulose, sintered polyethylene, and glass fibers. The one or more materials of the one or more fluid-transporting layers of the lateral flow assay region may be the same as or different from the one or more materials of the one or more fluid-transporting layers of the reagent delivery region.

In some embodiments, the one or more fluid-transporting layers comprise a plurality of fibers (e.g., woven or non-woven fabrics). In some embodiments, the one or more fluid-transporting layers comprise a plurality of pores. In some embodiments, pores and/or interstices between fibers may advantageously facilitate fluid transport (e.g., via capillary action). The pores may have any suitable average pore size. In certain embodiments, the plurality of pores has an average pore size of 30 μm or less, 25 μm or less, 20 μm or less, 15 μm or less, 10 μm or less, 5 μm or less, 2 μm or less, 1 μm or less, 0.9 μm or less, 0.8 μm or less, 0.7 μm or less, 0.6 μm or less, 0.5 μm or less, 0.4 μm or less, 0.3 μm or less, 0.2 μm or less, or 0.1 μm or less. In certain embodiments, the plurality of pores has an average pore size of at least 0.1 μm, at least 0.2 μm, at least 0.3 μm, at least 0.4 μm, at least 0.5 μm, at least 0.6 μm, at least 0.7 μm, at least 0.8 μm, at least 0.9 μm, at least 1 μm, at least 2 μm, at least 5 μm, at least 10 μm, at least 15 μm, at least 20 μm, at least 25 μm, or at least 30 μm. In some embodiments, the plurality of pores has an average pore size in a range from 0.1 μm to 0.5 μm, 0.1 μm to 1 μm, 0.1 μm to 5 pam, 0.1 μm to 10 μm, 0.1 μm to 15 μm, 0.1 μm to 20 μm, 0.1 μm to 25 μm, 0.1 μm to 30 μm, 0.5 μm to 1 μm, 0.5 μm to 5 μm, 0.5 μm to 10 μm, 0.5 μm to 15 μm, 0.5 μm to 20 μm, 0.5 μm to 25 μm, 0.5 μm to 30 μm, 1 μm to 5 μm, 1 μm to 10 μm, 1 μm to 15 μm, 1 μm to 20 μm, 1 μm to 25 μm, 1 μm to 30 μm, 5 μm to 10 μm, 5 μm to 15 μm, 5 μm to 20 μm, 5 μm to 25 μm, 5 μm to 30 μm, 10 μm to 15 μm, 10 μm to 20 μm, 10 μm to 25 μm, 10 μm to 30 μm, 15 μm to 20 μm, 15 μm to 25 μm, 15 μm to 30 μm, or 20 μm to 30 μm.

The one or more fluid-transporting layers may have any suitable porosity. In some embodiments, the one or more fluid-transporting layers have a porosity of at least 10%, at least 20%, at least 30%, at least 40%, at least 50%, or at least 60%. In some embodiments, the one or more fluid-transporting layers have a porosity in a range from 10% to 20%, 10% to 30%, 10% to 40%, 10% to 50%, 10% to 60%, 20% to 40%, 20% to 50%, 20% to 60%, 30% to 50%, 30% to 60%, 40% to 60%, or 50% to 60%.

In some embodiments, at least a portion of the fluidic contents of a reaction tube are transported through the lateral flow assay region via capillary action. In certain embodiments, the lateral flow assay region comprises a first sub-region (e.g., a sample pad) where the fluidic contents of the reaction tube are introduced to the lateral flow assay region.

In certain embodiments, the lateral flow assay region comprises a second sub-region (e.g., a particle conjugate pad) comprising a plurality of labeled particles. In some cases, the particles comprise gold nanoparticles (e.g., colloidal gold nanoparticles). The particles may be labeled with any suitable label. Non-limiting examples of suitable labels include biotin, streptavidin, fluorescein isothiocyanate (FITC), fluorescein amidite (FAM), fluorescein, and digoxigenin (DIG).

In certain embodiments, the lateral flow assay region comprises a third sub-region (e.g., a test pad) comprising one or more test lines. In some embodiments, a first test line comprises a capture reagent (e.g., an immobilized antibody) configured to detect a first target nucleic acid. In certain embodiments, the lateral flow assay region comprises one or more additional test lines. In some instances, each test line of the lateral flow assay region is configured to detect a different target nucleic acid. In some instances, two or more test lines of the lateral flow assay region are configured to detect the same target nucleic acid. The test line(s) may have any suitable shape or pattern (e.g., one or more straight lines, curved lines, dots, squares, check marks, x marks).

In certain embodiments, the third sub-region (e.g., the test pad) of the lateral flow assay region comprises one or more control lines. In certain instances, a first control line is a human (or animal) nucleic acid control line. In some embodiments, for example, the human (or animal) nucleic acid control line is configured to detect a nucleic acid (e.g., RNase P) that is generally present in all humans (or animals). In some cases, the human (or animal) nucleic acid control line becoming detectable indicates that a human (or animal) sample was successfully collected, nucleic acids from the sample were amplified, and the amplicons were transported through the lateral flow assay region. In certain instances, a first control line is a lateral flow control line. In some cases, the lateral flow control line becoming detectable indicates that a liquid was successfully transported through the lateral flow assay region. In some embodiments, the lateral flow assay region comprises two or more control lines. The control line(s) may have any suitable shape or pattern (e.g., one or more straight lines, curved lines, dots, squares, check marks, x marks). In some instances, for example, the lateral flow assay region comprises a human (or animal) nucleic acid control line and a lateral flow control line. In certain embodiments, the lateral flow assay region comprises a fourth sub-region (e.g., a wicking area) to absorb fluid flowing through the lateral flow assay region.

As an illustrative example, a fluidic sample comprising an amplicon labeled with biotin and FITC may be introduced into a lateral flow assay region (e.g., through a sample pad of a lateral flow assay region). In some embodiments, as the labeled amplicon is transported through the lateral flow assay region (e.g., through a particle conjugate pad of the lateral flow assay region), a gold nanoparticle labeled with streptavidin may bind to the biotin label of the amplicon. In some cases, the lateral flow assay region (e.g., a test pad of the lateral flow assay region) may comprise a first test line comprising an anti-FITC antibody. In some embodiments, the gold nanoparticle-amplicon conjugate may be captured by the anti-FITC antibody, and an opaque band may develop as additional gold nanoparticle-amplicon conjugates are captured by the anti-FITC antibodies of the first test line. In some cases, the lateral flow assay region (e.g., a test pad of the lateral flow assay region) further comprises a first lateral flow control line comprising biotin. In some embodiments, excess gold nanoparticles labeled with streptavidin (i.e., gold nanoparticles that were not conjugated to an amplicon) transported through the lateral flow assay region may bind to the biotin of the first lateral flow control line, demonstrating that liquid was successfully transported to the first lateral flow control line.

The lateral flow assay region may have any suitable dimensions. In some embodiments, the lateral flow assay region has a length of at least 1 cm, at least 2 cm, at least 3 cm, at least 4 cm, at least 5 cm, at least 6 cm, at least 7 cm, at least 8 cm, at least 9 cm, or at least 10 cm. In some embodiments, the lateral flow assay region has a length of 10 cm or less, 9 cm or less, 8 cm or less, 7 cm or less, 6 cm or less, 5 cm or less, 4 cm or less, 3 cm or less, 2 cm or less, or 1 cm or less. In some embodiments, the lateral flow assay region has a length in a range from 1 cm to 2 cm, 1 cm to 3 cm, 1 cm to 4 cm, 1 cm to 5 cm, 1 cm to 6 cm, 1 cm to 7 cm, 1 cm to 8 cm, 1 cm to 9 cm, 1 cm to 10 cm, 2 cm to 5 cm, 2 cm to 8 cm, 2 cm to 10 cm, 3 cm to 5 cm, 3 cm to 8 cm, 3 cm to 10 cm, 4 cm to 8 cm, 4 cm to 10 cm, 5 cm to 8 cm, 5 cm to 10 cm, or 8 cm to 10 cm.

In some embodiments, the sample pad of the lateral flow assay region has a length of 10 mm or less, 9 mm or less, 8 mm or less, 7 mm or less, 6 mm or less, 5 mm or less, 4 mm or less, 3 mm or less, 2 mm or less, or 1 mm or less. In certain embodiments, the sample pad of the lateral flow assay region has a length in a range from 1 mm to 2 mm, 1 mm to 3 mm, 1 mm to 4 mm, 1 mm to 5 mm, 1 mm to 6 mm, 1 mm to 7 mm, 1 mm to 8 mm, 1 mm to 9 mm, 1 mm to 10 mm, 2 mm to 5 mm, 2 mm to 8 mm, 2 mm to 10 mm, 5 mm to 8 mm, 5 mm to 10 mm, or 8 mm to 10 mm.

In some embodiments, at least a portion of the sample pad is configured to be inserted into a reaction tube, and the length of the sample pad is less than an initial depth of fluidic contents of the reaction tube. In certain embodiments, the length of the sample pad is 60% or less, 50% or less, 40% or less, 30% or less, 20% or less, 10% or less, 5% or less, or 1% or less of the initial depth of fluidic contents of the reaction tube. In some embodiments, the length of the sample pad is 1-5%, 1-10%, 1-20%, 1-30%, 1-40%, 1-50%, 1-60%, 5-10%, 5-20%, 5-30%, 5-40%, 5-50%, 5-60%, 10-20%, 10-30%, 10-40%, 10-50%, 10-60%, 20-50%, 20-60%, 30-50%, 30-60%, 40-60%, or 50-60% of the initial depth of fluidic contents of the reaction tube. In some instances, the sample pad is at least partially submerged in the fluidic contents of the reaction tube (e.g., the one or more liquids of the reaction tube) after insertion into the reaction tube (e.g., after one or more movements of the inner component relative to the outer component). In some instances, the sample pad is fully submerged in the fluidic contents of the reaction tube (e.g., the one or more liquids of the reaction tube) after insertion into the reaction tube (e.g., after one or more movements of the inner component relative to the outer component).

### Additional Components

In some embodiments, a diagnostic device comprises a removable cap covering at least a portion of the sample-collecting component. In some embodiments, the removable cap covers at least the swab element of the sample-collecting component. In some cases, the presence of the removable cap may ensure that the sample-collecting component is sterile until use.

In some embodiments, the removable cap comprises one or more protruding elements. In some embodiments, the removable cap comprises at least 1, at least 2, at least 3, a least 4, at least 5, at least 6, at least 7, at least 8, at least 9, or at least 10 protruding elements. Each protruding element may have any suitable shape. In some embodiments, the one or more protruding elements prevent the removable cap (and/or the diagnostic device to which it is attached) from being inserted into a reaction tube and/or a heating unit. In some embodiments, for example, a maximum diameter of the removable cap (including protruding elements) is greater than a maximum diameter of a reaction tube and/or a maximum diameter of an opening of a heating unit. In some embodiments, the removable cap is configured to hold a reaction tube.

In some embodiments, a diagnostic device comprises one or more safety clips maintaining a certain configuration of two or more components of the diagnostic device. For example, in some embodiments, a safety clip maintains an inner component and an outer component of the diagnostic device in a particular configuration and prevents the inner component from moving relative to the outer component until the safety clip is removed. In certain embodiments, the safety clip prevents the inner component from being pushed a first distance into an outer component. In certain embodiments, the safety clip prevents the inner component from being rotated relative to an outer component. In some cases, the safety clip ensures that a component of the diagnostic device is not accidentally and/or prematurely moved. In some embodiments, a diagnostic device comprises a plurality of safety clips. In some embodiments, a diagnostic device comprises at least 2, at least 3, at least 4, or at least 5 safety clips. In some embodiments, each safety clip prevents a different movement of a component of the diagnostic device. In some embodiments, two or more safety clips prevent the same movement of a component of the diagnostic device.

In some embodiments, the inner component is movable relative to the outer component. In some embodiments, the inner component and the outer component are configured such that a user can perform a first action that moves the inner component relative to the outer component. In some cases, the first action causes a first portion of the inner component (e.g., a reagent delivery region of a substrate) to be in physical contact with fluidic contents of a reaction tube. In some cases, the first action comprises pushing the inner component a first distance into the outer component. In some cases, the first distance is at least 5 mm, at least 10 mm, at least 15 mm, at least 20 mm, at least 25 mm, at least 30 mm, at least 35 mm, at least 40 mm, at least 45 mm, or at least 50 mm. In some cases, the first distance is in a range from 5 mm to 10 mm, 5 mm to 15 mm, 5 mm to 20 mm, 5 mm to 25 mm, 5 mm to 30 mm, 5 mm to 35 mm, 5 mm to 40 mm, 5 mm to 45 mm, 5 mm to 50 mm, 10 mm to 15 mm, 10 mm to 20 mm, 10 mm to 25 mm, 10 mm to 30 mm, 10 mm to 35 mm, 10 mm to 40 mm, 10 mm to 45 mm, 10 mm to 50 mm, 20 mm to 30 mm, 20 mm to 40 mm, 20 mm to 50 mm, 30 mm to 40 mm, 30 mm to 50 mm, or 40 mm to 50 mm.

In some embodiments, the first action comprises rotating the inner component relative to the outer component. In some embodiments, the first action comprises rotating the inner component relative to the outer component by at least 30 degrees, at least 45 degrees, at least 60 degrees, at least 90 degrees, at least 120 degrees, at least 180 degrees, at least 270 degrees, or at least 360 degrees. In some embodiments, the first action comprises rotating the inner component relative to the outer component by an amount in the range of 30−60°, 30-90°, 30-120°, 30-180°, 30-270°, 30-360°, 45-90°, 45-120°, 45-180°, 45-270°, 45-360°, 90-120°, 90-180°, 90-270°, 90-360°, 120-180°, 120-270°, 120-360°, 180-270°, 180-360°, or 270-360°.

In some embodiments, two or more actions are required to cause the first portion of the inner component (e.g., a reagent delivery region of a substrate) to be in physical contact with fluidic contents of a reaction tube. The two or more actions may comprise any combination of pushing, pulling, and/or rotating the inner component relative to the outer component.

In some embodiments, the inner component and the outer component are configured such that a user can perform a second action that moves the inner component relative to the outer component. In some embodiments, the second action causes a second portion of the inner component (e.g., a lateral flow assay region of a substrate) to be in physical contact with fluidic contents of a reaction tube. In some embodiments, the second action comprises pushing the inner component a second distance into the outer component. In some cases, the second distance is at least 1 mm, at least 2 mm, at least 5 mm, at least 10 mm, at least 15 mm, at least 20 mm, at least 25 mm, or at least 30 mm. In some cases, the second distance is 30 mm or less, 25 mm or less, 20 mm or less, 15 mm or less, 10 mm or less, 5 mm or less, 2 mm or less, or 1 mm or less. In some embodiments, the second distance is in a range from 1 mm to 2 mm, 1 mm to 5 mm, 1 mm to 10 mm, 1 mm to 15 mm, 1 mm to 20 mm, 1 mm to 25 mm, 1 mm to 30 mm, 5 mm to 10 mm, 5 mm to 15 mm, 5 mm to 20 mm, 5 mm to 25 mm, 5 mm to 30 mm, 10 mm to 20 mm, 10 mm to 25 mm, 10 mm to 30 mm, or 20 mm to 30 mm.

In some embodiments, the second action comprises rotating the inner component relative to the outer component. In some embodiments, the second action comprises rotating the inner component relative to the outer component by at least 30 degrees, at least 45 degrees, at least 60 degrees, at least 90 degrees, at least 120 degrees, at least 180 degrees, at least 270 degrees, or at least 360 degrees. In some embodiments, the second action comprises rotating the inner component relative to the outer component by an amount in the range of 30−60°, 30-90°, 30-120°, 30-180°, 30-270°, 30-360°, 45-90°, 45-120°, 45-180°, 45-270°, 45-360°, 90-120°, 90-180°, 90-270°, 90-360°, 120-180°, 120-270°, 120-360°, 180-270°, 180-360°, or 270-360°.

In some embodiments, two or more actions are required to cause the second portion of the inner component (e.g., a lateral flow assay region of a substrate) to be in physical contact with fluidic contents of a reaction tube. The two or more actions may comprise any combination of pushing, pulling, and/or rotating the inner component relative to the outer component.

### Diagnostic Test Kit

According to some embodiments, a diagnostic test kit is described. The kit may comprise a diagnostic device described herein and one or more additional components. In some embodiments, for example, the diagnostic test kit further comprises a reaction tube. In some embodiments, the diagnostic test kit further comprises a heating unit.

In some embodiments, the diagnostic test kit comprises a reaction tube. In some embodiments, the reaction tube has a partially or wholly removable cap. The reaction tube may be any reaction tube (e.g., an Eppendorf tube) capable of containing an amount of liquid. In some embodiments, the reaction tube is configured to hold a volume of at least 5 μL, at least 10 μL, at least 15 μL, at least 20 μL, at least 25 μL, at least 30 μL, at least 40 μL, at least 50 μL, at least 60 μL, at least 70 μL, at least 80 μL, at least 90 μL, at least 100 μL, at least 150 μL, at least 200 μL, at least 250 μL, at least 300 μL, at least 400 μL, at least 500 μL, at least 600 μL, at least 700 μL, at least 800 μL, at least 900 μL, at least 1 mL, at least 1.5 mL, or at least 2 mL. In some embodiments, the reaction tube is configured to hold a volume in a range from 5 μL to 10 μL, 5 μL to 20 μL, 5 μL to 50 μL, 5 μL to 70 μL, 5 μL to 100 μL, 5 μL to 200 μL, 5 μL to 500 μL, 5 μL to 1 mL, 5 μL to 1.5 mL, 5 μL to 2 mL, 10 μL to 20 μL, 10 μL to 50 μL, 10 μL to 70 μL, 10 μL to 100 μL, 10 μL to 200 μL, 10 μL to 500 μL, 10 μL to 1 mL, 10 μL to 1.5 mL, 10 μL to 2 mL, 20 μL to 50 μL, 20 μL to 70 μL, 20 μL to 100 μL, 20 μL to 200 μL, 20 μL to 500 μL, 20 μL to 1 mL, 20 μL to 1.5 mL, 20 μL to 2 mL, 50 μL to 70 μL, 50 μL to 100 μL, 50 μL to 200 μL, 50 μL to 500 μL, 50 μL to 1 mL, 50 μL to 1.5 mL, 50 μL to 2 mL, 70 μL to 100 μL, 70 μL to 200 μL, 70 μL to 500 μL, 70 μL to 1 mL, 70 μL to 1.5 mL, 70 μL to 2 mL, 100 μL to 200 μL, 100 μL to 500 μL, 100 μL to 1 mL, 100 μL to 1.5 mL, 100 μL to 2 mL, 200 μL to 500 μL, 200 μL to 1 mL, 200 μL to 1.5 mL, 200 μL to 2 mL, 500 μL to 1 mL, 500 μL to 1.5 mL, 500 μL to 2 mL, 1 mL to 1.5 mL, or 1 mL to 2 mL.

In some embodiments, the reaction tube contains an amount of one or more liquids (i.e., fluidic contents). In certain embodiments, the fluidic contents of the reaction tube have a volume sufficient to facilitate fluid flow through a lateral flow strip. In some embodiments, the fluidic contents of the reaction tube have an initial volume of at least 5 μL, at least 10 μL, at least 15 μL, at least 20 μL, at least 25 μL, at least 30 μL, at least 40 μL, at least 50 μL, at least 60 μL, at least 70 μL, at least 80 μL, at least 90 μL, at least 100 μL, at least 150 μL, at least 200 μL, at least 250 μL, at least 300 μL, at least 400 μL, at least 500 μL, at least 600 μL, at least 700 μL, at least 800 μL, at least 900 μL, at least 1 mL, at least 1.5 mL, or at least 2 mL. In some embodiments, the fluidic contents of the reaction tube have an initial volume in a range from 5 μL to 10 μL, 5 μL to 20 μL, 5 μL to 50 μL, 5 μL to 70 μL, 5 μL to 100 μL, 5 μL to 200 μL, 5 μL to 500 μL, 5 μL to 1 mL, 5 μL to 1.5 mL, 5 μL to 2 mL, 10 μL to 20 μL, 10 μL to 50 μL, 10 μL to 70 μL, 10 μL to 100 μL, 10 μL to 200 μL, 10 μL to 500 μL, 10 μL to 1 mL, 10 μL to 1.5 mL, 10 μL to 2 mL, 20 μL to 50 μL, 20 μL to 70 μL, 20 μL to 100 μL, 20 μL to 200 μL, 20 μL to 500 μL, 20 μL to 1 mL, 20 μL to 1.5 mL, 20 μL to 2 mL, 50 μL to 70 μL, 50 μL to 100 μL, 50 μL to 200 μL, 50 μL to 500 μL, 50 μL to 1 mL, 50 μL to 1.5 mL, 50 μL to 2 mL, 70 μL to 100 μL, 70 μL to 200 μL, 70 μL to 500 μL, 70 μL to 1 mL, 70 μL to 1.5 mL, 70 μL to 2 mL, 100 μL to 200 μL, 100 μL to 500 μL, 100 μL to 1 mL, 100 μL to 1.5 mL, 100 μL to 2 mL, 200 μL to 500 μL, 200 μL to 1 mL, 200 μL to 1.5 mL, 200 μL to 2 mL, 500 μL to 1 mL, 500 μL to 1.5 mL, 500 μL to 2 mL, 1 mL to 1.5 mL, or 1 mL to 2 mL.

In some embodiments, the fluidic contents of the reaction tube have an initial depth of at least 6 mm, at least 7 mm, at least 8 mm, at least 9 mm, at least 10 mm, at least 11 mm, at least 12 mm, at least 13 mm, at least 14 mm, at least 15 mm, at least 16 mm, at least 17 mm, at least 18 mm, at least 19 mm, or at least 20 mm. In some embodiments, the fluidic contents of the reaction tube have an initial depth in a range from 6 mm to 8 mm, 6 mm to 10 mm, 6 mm to 12 mm, 6 mm to 15 mm, 6 mm to 18 mm, 6 mm to 20 mm, 8 mm to 10 mm, 8 mm to 12 mm, 8 mm to 15 mm, 8 mm to 18 mm, 8 mm to 20 mm, 10 mm to 15 mm, 10 mm to 18 mm, 10 mm to 20 mm, or 15 mm to 20 mm.

In some embodiments, the fluidic contents of the reaction tube comprise a reaction buffer. In certain instances, the reaction buffer comprises one or more buffers. Non-limiting examples of suitable buffers include phosphate-buffered saline (“PBS”) and Tris. In certain instances, the reaction buffer comprises one or more salts. Non-limiting examples of suitable salts include magnesium sulfate, magnesium acetate tetrahydrate, potassium acetate, potassium chloride, and ammonium sulfate. In some embodiments, the concentration of at least one of the one or more salts (and, in some cases, each of the one or more salts) is at least 1 mM, at least 2 mM, at least 3 mM, at least 4 mM, at least 5 mM, at least 6 mM, at least 7 mM, at least 8 mM, at least 9 mM, at least 10 mM, at least 20 mM, at least 30 mM, at least 40 mM, at least 50 mM, or at least 100 mM. In certain embodiments, the concentration of at least one of the one or more salts (and, in some cases, each of the one or more salts) is in a range from 1 mM to 5 mM, 1 mM to 8 mM, 1 mM to 10 mM, 1 mM to 20 mM, 1 mM to 50 mM, 1 mM to 80 mM, 1 mM to 100 mM, 5 mM to 8 mM, 5 mM to 10 mM, 5 mM to 20 mM, 5 mM to 50 mM, 5 mM to 80 mM, 5 mM to 100 mM, 8 mM to 10 mM, 8 mM to 20 mM, 8 mM to 50 mM, 8 mM to 80 mM, 8 mM to 100 mM, 10 mM to 20 mM, 10 mM to 50 mM, 10 mM to 80 mM, 10 mM to 100 mM, 20 mM to 50 mM, 20 mM to 80 mM, 20 mM to 100 mM, 50 mM to 80 mM, 50 mM to 100 mM, or 80 mM to 100 mM.

In some embodiments, the reaction buffer comprises Tween (e.g., Tween 20, Tween 80). In some embodiments, the reaction buffer comprises an RNase inhibitor. In certain instances, Tween and/or RNase inhibitor may facilitate cell lysis.

In one non-limiting embodiment, the reaction buffer comprises 20 mM Tris-HCl, 0.1% (v/v) Tween 20, 8 mM magnesium sulfate, 10 mM ammonium sulfate, and 50 mM potassium chloride. In another non-limiting embodiment, the reaction buffer comprises 25 mM Tris buffer, 5% (w/v) poly(ethylene glycol) 35,000 kDa, 14 mM magnesium acetate tetrahydrate, 100 mM potassium acetate, and greater than 85% volume nuclease free water.

In some embodiments, the reaction buffer has a relatively neutral pH. In some embodiments, the reaction buffer has a pH in a range from 5.0 to 6.0, 5.0 to 7.0, 5.0 to 8.0, 5.0 to 9.0, 5.0 to 10.0, 6.0 to 7.0, 6.0 to 8.0, 6.0 to 9.0, 6.0 to 10.0, 7.0 to 8.0, 7.0 to 9.0, 7.0 to 10.0, 8.0 to 9.0, 8.0 to 10.0, or 9.0 to 10.0.

### Heating Unit

In some embodiments, a diagnostic test kit comprises a heating unit. The heating unit may be any device capable of heating fluidic contents of a reaction tube. In certain embodiments, the heating unit is a battery-powered heat source, a USB-powered heat source, a hot plate, a heating coil, or a hot water bath. In some embodiments, the heating unit is contained within a thermally-insulated housing to ensure user safety. In some embodiments, the heating unit is an off-the-shelf consumer-grade device.

In some embodiments, the heating unit is configured to heat fluidic contents of a reaction tube to a temperature of at least 37° C., at least 40° C., at least 50° C., at least 55° C., at least 60° C., at least 63.5° C., at least 65° C., at least 70° C., at least 75° C., at least 80° C., at least 85° C., at least 90° C., or at least 100° C. In some embodiments, the heating unit is configured to heat fluidic contents of a reaction tube to a temperature in a range from 37° C. to 50° C., 37° C. to 60° C., 37° C. to 63.5° C., 37° C. to 65° C., 37° C. to 70° C., 37° C. to 80° C., 37° C. to 90° C., 37° C. to 100° C., 50° C. to 60° C., 50° C. to 65° C., 50° C. to 70° C., 50° C. to 80° C., 50° C. to 90° C., 50° C. to 100° C., 55° C. to 65° C., 55° C. to 70° C., 55° C. to 75° C., 55° C. to 80° C., 55° C. to 90° C., 55° C. to 100° C., 60° C. to 70° C., 60° C. to 75° C., 60° C. to 80° C., 60° C. to 90° C., 60° C. to 100° C., 63.5° C. to 75° C., 63.5° C. to 80° C., 63.5° C. to 90° C., 63.5° C. to 100° C., 65° C. to 75° C., 65° C. to 80° C., 65° C. to 90° C., 65° C. to 100° C., 70° C. to 80° C., 70° C. to 90° C., 70° C. to 100° C., 80° C. to 90° C., 80° C. to 100° C., or 90° C. to 100° C.

In some embodiments, the heating unit is configured to heat fluidic contents of a reaction tube to a desired temperature for at least 1 minute, at least 2 minutes, at least 3 minutes, at least 4 minutes, at least 5 minutes, at least 10 minutes, at least 15 minutes, at least 20 minutes, at least 30 minutes, at least 45 minutes, at least 60 minutes, or at least 90 minutes. In certain embodiments, the heating unit is configured to heat fluidic contents of a reaction tube to a desired temperature for a time in a range from 1 to 3 minutes, 1 to 5 minutes, 1 to 10 minutes, 1 to 15 minutes, 1 to 20 minutes, 1 to 30 minutes, 1 to 40 minutes, 1 to 50 minutes, 1 to 60 minutes, 5 minutes to 10 minutes, 5 minutes to 15 minutes, 5 minutes to 20 minutes, 5 minutes to 30 minutes, 5 minutes to 45 minutes, 5 minutes to 60 minutes, 5 minutes to 90 minutes, 10 minutes to 15 minutes, 10 minutes to 20 minutes, 10 minutes to 30 minutes, 10 minutes to 45 minutes, 10 minutes to 60 minutes, 10 minutes to 90 minutes, 15 minutes to 30 minutes, 15 minutes to 45 minutes, 15 minutes to 60 minutes, 15 minutes to 90 minutes, 30 minutes to 45 minutes, 30 minutes to 60 minutes, 30 minutes to 90 minutes, or 60 minutes to 90 minutes.

In some embodiments, the heating unit comprises at least two temperature zones. In certain instances, for example, the heating unit is an off-the-shelf consumer-grade heating coil connected to a microcontroller that is used to switch between two temperature zones. In some embodiments, the first temperature zone is in a range from 60° C. to 100° C., 60° C. to 90° C., 60° C. to 80° C., 60° C. to 70° C., or 60° C. to 65° C. In certain cases, the first temperature zone has a temperature of approximately 65° C. In some embodiments, the second temperature zone is in a range from 30° C. to 40° C. In certain cases, the second temperature zone has a temperature of approximately 37° C.

### Instructions & Software

In some embodiments, a diagnostic test kit comprises instructions for using a diagnostic device and/or performing a diagnostic test method. The instructions may include instructions for the use, assembly, and/or storage of the diagnostic device and any other components associated with the kit. The instructions may be provided in any form recognizable by one of ordinary skill in the art as a suitable vehicle for containing such instructions. For example, the instructions may be written or published, verbal, audible (e.g., telephonic), digital, optical, visual (e.g., videotape, DVD, etc.) or electronic communications (including Internet or web-based communications).

In some embodiments, the instructions are provided as part of a software-based application. In certain cases, the application can be downloaded to a smartphone or device, and then guides a user through steps to use the diagnostic device. In some embodiments, the application may validate that a diagnostic test was performed correctly.

In some embodiments, a diagnostic test kit or diagnostic device comprises or is associated with software to read and/or analyze test results. In some embodiments, a device (e.g., a camera, a smartphone) is used to generate an image of a test result (e.g., one or more lines detectable through openings in the inner and outer components of the device). In certain cases, a machine vision software application is employed to evaluate the image and provide a positive or negative test result. That result may be communicated directly to a user or to a medical professional. In some cases, the test result may be further communicated to a remote database server. In some embodiments, the remote database server stores test results as well as user information. For example, the remote database server may store at least one of name, social security number, date of birth, address, phone number, email address, medical history, and medications.

In some embodiments, the remote database server may track and monitor locations of users (e.g., using smartphones or remote devices with tracking capabilities). In some cases, the remote database server can be used to notify individuals who come into contact with or within a certain distance of any user who has tested positive for a particular illness (e.g., COVID-19). In some cases, a user's test results, information, and/or location may be communicated to state and/or federal health agencies.

### Diagnostic Test Method

Some embodiments are directed to a diagnostic test method. In some embodiments, the diagnostic test method comprises collecting a sample from a subject (e.g., a human subject, an animal subject). In some embodiments, collecting the sample from the subject comprises inserting at least a portion of a sample-collecting component (e.g., a swab element) into a cavity of the subject. In certain embodiments, the cavity is a nasal cavity, an oral cavity, a vaginal cavity, an anal cavity, or an ear canal. In certain embodiments, collecting the sample from the subject comprises collecting a bodily secretion (e.g., a nasal secretion, an oral secretion, a genital secretion) from the subject. In some embodiments, the sample comprises a nasal secretion (e.g., mucus), an oral secretion (e.g., saliva), a genital secretion, a cell scraping (e.g., a scraping from the mouth or interior cheek), blood, urine, exhaled breath particles, and/or other bodily fluids. In some embodiments, collecting the sample comprises a user self collecting the sample. In some embodiments, collecting the sample comprises an individual collecting the sample from a separate subject. That is, the sample may be self-collected by the subject or may be collected by another individual (e.g., a family member, a friend, a coworker, a health care professional).

In certain embodiments, the nasal secretion is an anterior nares specimen. In some embodiments, an anterior nares specimen is collected from a subject by inserting at least a portion of a sample-collecting component (e.g., a swab element) of a diagnostic device into one or both nostrils of the subject for a period of time. In some embodiments, the period of time is at least 5 seconds, at least 10 seconds, at least 20 seconds, or at least 30 seconds. In some embodiments, the period of time is 30 seconds or less, 20 seconds or less, 10 seconds or less, or 5 seconds or less. In some embodiments, the period of time is in a range from 5 seconds to 10 seconds, 5 seconds to 20 seconds, 5 seconds to 30 seconds, 10 seconds to 20 seconds, or 10 seconds to 30 seconds.

In some embodiments, the sample comprises a cell scraping. The cell scraping may be collected using a brush or scraping device formulated for this purpose.

In some embodiments, the sample comprises saliva. In certain cases, the volume of saliva in the sample is at least 1 mL, at least 1.5 mL, at least 2 mL, at least 2.5 mL, at least 3 mL, at least 3.5 mL, or at least 4 mL. In some embodiments, the volume of saliva in the sample is in a range from 1 mL to 2 mL, 1 mL to 3 mL, 1 mL to 4 mL, or 2 mL to 4 mL. Saliva has been found to have a mean concentration of SARS-Cov-2 RNA of 5 fM (Kai-Wang To et al., 2020) an amount that is detectable by any one of the methods described herein.

In some embodiments, the concentration of pathogen RNA or DNA (e.g., COVID-19 RNA) in a sample is at least 5 aM, at least 10 aM, at least 15 aM, at least 20 aM, at least 25 aM, at least 30 aM, at least 35 aM, at least 40 aM, at least 50 aM, at least 75 aM, at least 100 aM, at least 150 aM, at least 200 aM, at least 300 aM, at least 400 aM, at least 500 aM, at least 600 aM, at least 700 aM, at least 800 aM, at least 900 aM, at least 1 fM, at least 5 fM, at least 10 fM, at least 15 fM, at least 20 fM, at least 25 fM, at least 30 fM, at least 35 fM, at least 40 fM, at least 50 fM, at least 75 fM, at least 100 fM, at least 150 fM, at least 200 fM, at least 300 fM, at least 400 fM, at least 500 fM, at least 600 fM, at least 700 fM, at least 800 fM, at least 900 fM, at least 1 pM, at least 5 pM, or at least 10 pM. In some embodiments, the concentration of pathogen RNA or DNA (e.g., COVID-19 RNA) is 10 pM or less, 5 pM or less, 1 pM or less, 500 fM or less, 100 fM or less, 50 fM or less, 10 fM or less, 1 fM or less, 500 aM or less, 100 aM or less, 50 aM or less 10 aM or less, or 5 aM or less. In some embodiments, the concentration of pathogen RNA or DNA (e.g., COVID-19 RNA) in the sample is in a range from 5 aM to 50 aM, 5 aM to 100 aM, 5 aM to 500 aM, 5 aM to 1 fM, 5 aM to 10 fM, 5 aM to 50 fM, 5 aM to 100 fM, 5 aM to 500 fM, 5 aM to 1 pM, 5 aM to 10 pM, 10 aM to 50 aM, 10 aM to 100 aM, 10 aM to 500 aM, 10 aM to 1 fM, 10 aM to 10 fM, 10 aM to 50 fM, 10 aM to 100 fM, 10 aM to 500 fM, 10 aM to 1 pM, 10 aM to 10 pM, 100 aM to 500 aM, 100 aM to 1 fM, 100 aM to 10 fM, 100 aM to 50 fM, 100 aM to 100 fM, 100 aM to 500 fM, 100 aM to 1 pM, 100 aM to 10 pM, 1 fM to 10 fM, 1 fM to 50 fM, 1 fM to 100 fM, 1 fM to 500 fM, 1 fM to 1 pM, 1 fM to 10 pM, 5 fM to 10 fM, 5 fM to 50 fM, 5 fM to 100 fM, 5 fM to 500 fM, 5 fM to 1 pM, 5 fM to 10 pM, 10 fM to 100 fM, 10 fM to 500 fM, 10 fM to 1 pM, 10 fM to 10 pM, 100 fM to 500 fM, 100 fM to 1 pM, 100 fM to 10 pM, or 1 pM to 10 pM.

In some embodiments, the sample is collected from a subject who is suspected of having the disease(s) the test screens for, such as a coronavirus (e.g., COVID-19) and/or influenza (e.g., influenza A or influenza B). Other indications, as described herein, are also envisioned. In some embodiments, a subject (e.g., a human subject) is asymptomatic. In some embodiments, a subject (e.g., a human subject) presents with one or more symptoms of the disease(s). Symptoms of coronaviruses (e.g., COVID-19) include, but are not limited to, fever, cough (e.g., dry cough), generalized fatigue, sore throat, runny nose, nasal congestion, muscle aches, and difficulty breathing (shortness of breath). Symptoms of influenza include, but are not limited to, fever, chills, muscle aches, cough, congestion, runny nose, headaches, and generalized fatigue. In some embodiments, the subject has had contact within a certain period of time (e.g., the past 14 days) with a person who has tested positive for the disease.

According to some embodiments, the diagnostic test method further comprises amplifying one or more target nucleic acids within the sample.

In some embodiments, the diagnostic test method comprises, after collecting the sample using at least a portion of a sample-collecting component (e.g., at least a portion of a swab element) of a diagnostic device, inserting at least a portion of the sample-collecting component (e.g., at least a portion of the swab element) into a reaction tube. In some embodiments, the method comprises moving an inner component of the diagnostic device relative to an outer component of the diagnostic device in a first movement such that at least a first portion of the inner component is exposed to fluidic contents of the reaction tube. In certain embodiments, the first portion of the inner component is a reagent delivery region of a substrate. In some instances, the reagent delivery region comprises one or more reagents. The one or more reagents may comprise lysis reagents, reverse transcription reagents, nucleic acid amplification reagents (e.g., LAMP reagents, RPA reagents, tHDA reagents, NEAR reagents), and/or CRISPR/Cas detection reagents. In some cases, physical contact between the reagent delivery region of the substrate and fluidic contents of the reaction tube may dissolve the one or more reagents in the fluidic contents of the reaction tube.

In some cases, the diagnostic test method comprises heating the reaction tube according to a heating protocol (e.g., an amplification heating protocol). In some cases, the method does not require a step of heating the reaction tube. In such embodiments, the step of applying the heating protocol as described below would not be necessary for nucleic acid amplification and would not be performed.

In some embodiments, a heating protocol comprises heating the reaction tube at a first temperature for a first time period. In certain instances, the first temperature is at least 30° C., at least 32° C., at least 37° C., at least 40° C., at least 50° C., at least 55° C., at least 60° C., at least 63.5° C., at least 65° C., at least 70° C., at least 75° C., at least 80° C., or at least 90° C. In certain embodiments, the first temperature is in a range from 30° C. to 37° C., 30° C. to 40° C., 30° C. to 50° C., 30° C. to 60° C., 30° C. to 65° C., 30° C. to 70° C., 30° C. to 75° C., 30° C. to 80° C., 30° C. to 90° C., 37° C. to 50° C., 37° C. to 60° C., 37° C. to 63.5° C., 37° C. to 65° C., 37° C. to 70° C., 37° C. to 75° C., 37° C. to 80° C., 37° C. to 90° C., 50° C. to 60° C., 50° C. to 65° C., 50° C. to 70° C., 50° C. to 80° C., 50° C. to 90° C., 55° C. to 75° C., 55° C. to 90° C., 60° C. to 65° C., 60° C. to 70° C., 60° C. to 75° C., 60° C. to 90° C., 61° C. to 69° C., 62° C. to 68° C., 63° C. to 67° C., 64° C. to 66° C., 65° C. to 70° C., or 75° C. to 90° C. In certain instances, the first temperature is about 37° C.

In some embodiments, the first time period is 60 minutes or less, 45 minutes or less, 40 minutes or less, 35 minutes or less, 30 minutes or less, 25 minutes or less, 20 minutes or less, 15 minutes or less, 10 minutes or less, 5 minutes or less, 4 minutes or less, 3 minutes or less, 2 minutes or less, or about 1 minute. In some embodiments, the first time period is in a range from 1 minute to 3 minutes, 1 minute to 5 minutes, 1 minute to 10 minutes, 1 minute to 15 minutes, 1 minute to 20 minutes, 1 minute to 30 minutes, 1 minute to 40 minutes, 1 minute to 45 minutes, 1 minute to 50 minutes, 1 minute to 60 minutes, 3 minutes to 5 minutes, 3 minutes to 10 minutes, 3 minutes to 15 minutes, 3 minutes to 20 minutes, 3 minutes to 30 minutes, 3 minutes to 40 minutes, 3 minutes to 50 minutes, 3 minutes to 60 minutes, 5 minutes to 10 minutes, 5 minutes to 15 minutes, 5 minutes to 20 minutes, 5 minutes to 30 minutes, 5 minutes to 40 minutes, 5 minutes to 45 minutes, 5 minutes to 50 minutes, 5 minutes to 60 minutes, 10 minutes to 20 minutes, 10 minutes to 30 minutes, 10 minutes to 40 minutes, 10 minutes to 45 minutes, 10 minutes to 50 minutes, 10 minutes to 60 minutes, 15 minutes to 30 minutes, 15 minutes to 45 minutes, 15 minutes to 60 minutes, 20 minutes to 30 minutes, 20 minutes to 40 minutes, 20 minutes to 45 minutes, 20 minutes to 50 minutes, 20 minutes to 60 minutes, 25 minutes to 35 minutes, 30 minutes to 40 minutes, 30 minutes to 45 minutes, 30 minutes to 60 minutes, 40 minutes to 60 minutes, 45 minutes to 60 minutes, or 50 minutes to 60 minutes. In certain instances, the first time period is about 3 minutes.

In some embodiments, a heating protocol comprises heating the reaction tube at a second temperature for a second time period. In certain instances, the second temperature is at least 30° C., at least 32° C., at least 37° C., at least 50° C., at least 60° C., at least 63.5° C., at least 65° C., at least 70° C., at least 80° C., or at least 90° C. In certain instances, the second temperature is in a range from 30° C. to 37° C., 30° C. to 50° C., 30° C. to 60° C., 30° C. to 65° C., 30° C. to 70° C., 30° C. to 80° C., 30° C. to 90° C., 37° C. to 50° C., 37° C. to 60° C., 37° C. to 65° C., 37° C. to 70° C., 37° C. to 80° C., 37° C. to 90° C., 50° C. to 60° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C. In certain instances, the second temperature is about 63.5° C.

In certain instances, the second time period is at least 1 minute, at least 2 minutes, at least 3 minutes, at least 4 minutes, at least 5 minutes, at least 10 minutes, at least 15 minutes, at least 20 minutes, at least 30 minutes, at least 40 minutes, at least 45 minutes, at least 50 minutes, or at least 60 minutes. In some embodiments, the second time period is in a range from 1 minute to 3 minutes, 1 minute to 5 minutes, 1 minute to 10 minutes, 1 minute to 15 minutes, 1 minute to 20 minutes, 1 minute to 30 minutes, 1 minute to 40 minutes, 1 minute to 45 minutes, 1 minute to 50 minutes, 1 minute to 60 minutes, 3 minutes to 5 minutes, 3 minutes to 10 minutes, 3 minutes to 15 minutes, 3 minutes to 20 minutes, 3 minutes to 30 minutes, 3 minutes to 40 minutes, 3 minutes to 50 minutes, 3 minutes to 60 minutes, 5 minutes to 10 minutes, 5 minutes to 15 minutes, 5 minutes to 20 minutes, 5 minutes to 30 minutes, 5 minutes to 40 minutes, 5 minutes to 45 minutes, 5 minutes to 50 minutes, 5 minutes to 60 minutes, 10 minutes to 20 minutes, 10 minutes to 30 minutes, 10 minutes to 40 minutes, 10 minutes to 45 minutes, 10 minutes to 50 minutes, 10 minutes to 60 minutes, 15 minutes to 30 minutes, 15 minutes to 45 minutes, 15 minutes to 60 minutes, 20 minutes to 30 minutes, 20 minutes to 40 minutes, 20 minutes to 45 minutes, 20 minutes to 50 minutes, 20 minutes to 60 minutes, 25 minutes to 35 minutes, 30 minutes to 40 minutes, 30 minutes to 45 minutes, 30 minutes to 60 minutes, 40 minutes to 60 minutes, 45 minutes to 60 minutes, or 50 minutes to 60 minutes. In certain instances, the second time period is about 40 minutes. In some embodiments, a heating protocol does not comprise a second time period for heating.

In some embodiments, a heating protocol comprises heating the reaction tube at a third temperature for a third time period. In certain instances, the third temperature is at least 30° C., at least 32° C., at least 37° C., at least 50° C., at least 60° C., at least 63.5° C., at least 65° C., at least 70° C., at least 80° C., or at least 90° C. In certain instances, the third temperature is in a range from 30° C. to 37° C., 30° C. to 50° C., 30° C. to 60° C., 30° C. to 65° C., 30° C. to 70° C., 30° C. to 80° C., 30° C. to 90° C., 37° C. to 50° C., 37° C. to 60° C., 37° C. to 65° C., 37° C. to 70° C., 37° C. to 80° C., 37° C. to 90° C., 50° C. to 60° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C. In certain instances, the third time period is at least 1 minute, at least 2 minutes, at least 3 minutes, at least 4 minutes, at least 5 minutes, at least 10 minutes, at least 15 minutes, at least 20 minutes, at least 30 minutes, at least 45 minutes, or at least 60 minutes. In certain instances, the third time period is in a range from 1 to 3 minutes, 1 to 5 minutes, 1 to 10 minutes, 1 to 15 minutes, 1 to 20 minutes, 1 to 30 minutes, 1 to 45 minutes, 1 to 60 minutes, 3 to 5 minutes, 3 to 10 minutes, 3 to 15 minutes, 3 to 20 minutes, 3 to 30 minutes, 3 to 45 minutes, 3 to 60 minutes, 5 to 10 minutes, 5 to 15 minutes, 5 to 20 minutes, 5 to 30 minutes, 5 to 45 minutes, 5 to 60 minutes, 10 to 20 minutes, 10 to 30 minutes, 10 to 45 minutes, 10 to 60 minutes, 20 to 30 minutes, 20 to 45 minutes, 20 to 60 minutes, 30 to 45 minutes, 30 to 60 minutes, or 45 to 60 minutes. In some embodiments, a heating protocol does not comprise a third time period for heating.

In some embodiments, a heating protocol may comprise heating a sample at one or more additional temperatures for one or more additional time periods.

In one non-limiting example, the first temperature is in a range from 30° C. to 65° C. and the first time period is in a range from 1 minute to 5 minutes. For example, the first temperature may be approximately 37° C. and the first time period may be approximately 3 minutes. In another non-limiting example, the second temperature is in a range from 60° C. to 80° C. and the second time period is in a range from 30 minutes to 45 minutes. For example, the first temperature may be approximately 63.5° C. and the second time period may be approximately 40 minutes.

In some embodiments, the total heating time of a heating protocol is 90 minutes or less, 60 minutes or less, 50 minutes or less, 45 minutes or less, 40 minutes or less, 30 minutes or less, 20 minutes or less, 15 minutes or less, or 10 minutes or less. In some embodiments, the total heating time of the heating protocol is in a range from 10 to 20 minutes, 10 to 30 minutes, 10 to 40 minutes, 10 to 45 minutes, 10 to 50 minutes, 10 to 60 minutes, 10 to 90 minutes, 20 to 30 minutes, 20 to 40 minutes, 20 to 45 minutes, 20 to 50 minutes, 20 to 60 minutes, 20 to 90 minutes, 30 to 40 minutes, 30 to 45 minutes, 30 to 50 minutes, 30 to 60 minutes, 30 to 90 minutes, 40 to 50 minutes, 40 to 60 minutes, 45 to 60 minutes, 45 to 90 minutes, 50 to 60 minutes, 50 to 90 minutes, or 60 to 90 minutes.

In some embodiments, the diagnostic test method comprises moving an inner component of the diagnostic device relative to an outer component of the diagnostic device in a second movement such that at least a second portion of the inner component is exposed to fluidic contents of the reaction tube. In certain embodiments, the second portion of the inner component is a lateral flow assay region of the substrate. As discussed above, the lateral flow assay region may comprise one or more test lines configured to detect one or more target nucleic acids. In some embodiments, the lateral flow assay region comprises one or more control lines configured to confirm the presence of human (or animal) DNA in the sample and/or to confirm proper fluid flow through the lateral flow assay region.

In some embodiments, the diagnostic test method further comprises reading an indication of the presence or absence of a target nucleic acid in the sample. In some embodiments, if at least one control line and at least one test line are detectable, the test indicates that the sample is positive for a target nucleic acid. If at least one control line is detectable but no test lines are detectable, the test indicates that the sample is negative for a target nucleic acid. If no control lines or test lines are detectable, the test indicates that there was an error with collection of the sample and/or use of the diagnostic device.

In some embodiments, a user takes a picture of the lateral flow strip with a device (e.g., a camera, a smartphone). In some cases, the diagnostic device may contain markers that allow a mobile app to recognize the proper orientation of the image and provide feedback to the user. In some embodiments, a computer vision algorithm is used to confirm user interpretation of results. For example, a user may enter their results (e.g., on a “Record Results”) page, and the computer vision algorithm may confirm whether the band pattern in an image is consistent with the result entered by the user. If the algorithm determines that the band pattern differs from the result entered by the user, the user may be asked to double check that they entered the correct band pattern and is given the opportunity to redo the “Record Results” page. Alternatively, in some embodiments, interpretation of test results may be performed solely by the computer vision algorithm. The algorithm may provide an output informing the user whether the test result is positive, negative, or invalid.

In some embodiments, the total time for performing the diagnostic test method is about 100 minutes or less, about 90 minutes or less, about 80 minutes or less, about 75 minutes or less, about 70 minutes or less, about 65 minutes or less, about 60 minutes or less, about 50 minutes or less, 45 minutes or less, about 40 minutes or less, or about 30 minutes or less. In some embodiments, the total time for performing the diagnostic test method is in a range from 30 to 40 minutes, 30 to 45 minutes, 30 to 50 minutes, 30 to 60 minutes, 30 to 65 minutes, 30 to 70 minutes, 30 to 75 minutes, 30 to 80 minutes, 30 to 90 minutes, 30 to 100 minutes, 45 to 60 minutes, 45 to 65 minutes, 45 to 70 minutes, 45 to 75 minutes, 45 to 80 minutes, 45 to 90 minutes, 45 to 100 minutes, 60 to 70 minutes, 60 to 75 minutes, 60 to 80 minutes, 60 to 90 minutes, 60 to 100 minutes, 70 to 75 minutes, 70 to 80 minutes, 70 to 90 minutes, 70 to 100 minutes, 75 to 80 minutes, 75 to 90 minutes, 75 to 100 minutes, 80 to 90 minutes, or 80 to 100 minutes.

### Method of Manufacturing

Some embodiments are directed to a method of manufacturing a diagnostic device. In some embodiments, the method comprises providing an outer component. The outer component may be manufactured by injection molding, one or more additive manufacturing techniques (e.g., 3D printing), and/or one or more subtractive manufacturing techniques (e.g., laser cutting).

In some embodiments, the method comprises forming an inner component comprising a portion configured to detect a target nucleic acid. In certain embodiments, forming the inner component comprises adding one or more reagents to a reagent delivery region of a substrate. In some instances, the one or more reagents comprise one or more lysis reagents (e.g., enzymes, detergents). In some instances, the one or more reagents comprise reverse transcription reagents (e.g., reverse transcriptase). In some instances, the one or more reagents comprise nucleic acid amplification reagents (e.g., LAMP reagents, RPA reagents, NEAR reagents, tHDA reagents). In some embodiments, the one or more reagents comprise CRISPR/Cas detection reagents. In some embodiments, forming the inner component further comprises freeze drying, spraying, and/or wetting and drying the reagent delivery region of the substrate. In some embodiments, the one or more reagents may be lyophilized, crystallized (e.g., crystallized in a dried sugar solution), air jetted, and/or immersed in solution and placed in a drying chamber.

In certain embodiments, forming the inner component comprises adding one or more capture reagents (e.g., immobilized antibodies) to a lateral flow assay region of a substrate. In some cases, the one or more capture reagents are configured to capture one or more target nucleic acids. In some embodiments, the one or more target nucleic acids comprise a nucleic acid of a pathogen (e.g., a viral, bacterial, fungal protozoan, or parasitic pathogen). In some instances, the one or more target nucleic acids are nucleic acids of SARS-CoV-2 and/or an influenza virus. In some embodiments, forming the inner component further comprises freeze drying, spraying, and/or wetting and drying the lateral flow assay region of the substrate.

In some embodiments, the method comprises inserting the inner component within the outer component such that the inner component moves relative to the outer component.

Various inventive concepts may be embodied as one or more processes, of which examples have been provided. The acts performed as part of each process may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing,” “involving,” and variations thereof, is meant to encompass the items listed thereafter and additional items.

The terms “approximately,” “substantially,” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Having described several embodiments of the techniques described herein in detail, various modifications, and improvements will readily occur to those skilled in the art. Such modifications and improvements are intended to be within the spirit and scope of the disclosure. Accordingly, the foregoing description is by way of example only, and is not intended as limiting. The techniques are limited only as defined by the following claims and the equivalents thereto.

What is claimed is:

1. A diagnostic pen for detecting a first target nucleic acid, comprising:
an outer casing;
an inner member movable within the outer casing; and
a sample-collecting component attached to the outer casing or the inner member.

2. The diagnostic pen of claim 1, wherein the diagnostic pen has a length of about 25 cm or less and/or a maximum diameter of about 5 cm or less.

3. The diagnostic pen of claim 1, wherein the first target nucleic acid is a nucleic acid of SARS-CoV-2 or a variant thereof.

4. The diagnostic pen of claim 1, wherein the first target nucleic acid is a nucleic acid of an influenza virus.

5. The diagnostic pen of claim 1, wherein the inner member is configured to be pushed a first distance into the outer casing and/or rotated relative to the outer casing.

6. The diagnostic pen of claim 1, further comprising a first safety clip configured to prevent a first movement of the inner member relative to the outer casing until the first safety clip is removed.

7. The diagnostic pen of claim 1, wherein the inner member comprises a substrate comprising a reagent delivery region and a lateral flow assay region, wherein the reagent delivery region comprises one or more reagents and the lateral flow assay region is configured to detect the first target nucleic acid.

8. The diagnostic pen of claim 7, wherein the outer casing and the inner member each comprise an opening, wherein at least a portion of the substrate is visible when the opening of the outer casing and the opening of the inner member are aligned.

9. The diagnostic pen of claim 7, wherein a separation region is positioned between the reagent delivery region and the lateral flow assay region, wherein the separation region comprises one or more layers comprising one or more materials that do not allow fluid transport and does not comprise any materials that allow fluid transport.

10. The diagnostic pen of claim 7, wherein the one or more reagents comprise one or more lysis reagents, reverse transcription reagents, nucleic acid amplification reagents, and/or CRISPR/Cas detection reagents.

11. The diagnostic pen of claim 7, wherein the lateral flow assay region comprises a first test line comprising a first capture reagent configured to detect the first target nucleic acid, and wherein the lateral flow assay region further comprises one or more control lines.

12. The diagnostic pen of claim 11, wherein the lateral flow assay region comprises a second test line comprising a second capture reagent configured to detect a second target nucleic acid different from the first target nucleic acid.

13. A diagnostic kit, comprising:
the diagnostic pen of claim 1; and
a reaction tube comprising one or more liquids.

14. The diagnostic kit of claim 13, wherein the one or more liquids of the reaction tube have a volume in a range from 70 μL to 200 μL.

15. The diagnostic kit of claim 13, wherein the reagent delivery region has a length that is 10-40% of the initial depth of the one or more liquids of the reaction tube.

16. The diagnostic kit of claim 13, further comprising a heating unit configured to heat the reaction tube at a temperature in a range from 50° C. to 100° C.

17. A method of testing, comprising:
collecting a sample with a sample-collecting component, wherein the sample-collecting component is attached to an outer component and/or an inner component of a diagnostic device;
moving the inner component relative to the outer component in a first movement such that at least a first portion of the inner component is exposed to fluidic contents of a reaction tube; and
reading an indication of the presence or absence of a first target nucleic acid in the sample.

18. The method of claim 17, wherein the inner component of the diagnostic device comprises a substrate comprising a reagent delivery region and a lateral flow assay region.

19. The method of claim 17, wherein collecting the sample comprises inserting at least a portion of the sample-collecting element into a nasal or oral cavity of a subject.

20. The method of claim 17, further comprising heating the fluidic contents of the reaction tube to a temperature in a range from 50° C. to 100° C. for an amount of time in a range from 5 minutes to 60 minutes.


SEQUENCE LISTING (25 sequences)

SEQ ID NO: 1 - DNA, 18 bp, Artificial Sequence
  Synthetic
cggtggacaaattgtcac

SEQ ID NO: 2 - DNA, 22 bp, Artificial Sequence
  Synthetic
cttctctggatttaacacactt

SEQ ID NO: 3 - DNA, 28 bp, Artificial Sequence
  Synthetic
ttacaagcttaaagaatgtctgaacact

SEQ ID NO: 4 - DNA, 27 bp, Artificial Sequence
  Synthetic
ttgaatttaggtgaaacatttgtcacg

SEQ ID NO: 5 - DNA, 51 bp, Artificial Sequence
  Synthetic
tcagcacacaaagccaaaaatttatttttctgtgcaaaggaaattaaggag

SEQ ID NO: 6 - DNA, 49 bp, Artificial Sequence
  Synthetic
tattggtggagctaaacttaaagccttttctgtacaatccctttgagtg

SEQ ID NO: 7 - DNA, 47 bp, Artificial Sequence
  Synthetic
tcagcacacaaagccaaaaatttatctgtgcaaaggaaattaaggag

SEQ ID NO: 8 - DNA, 45 bp, Artificial Sequence
  Synthetic
tattggtggagctaaacttaaagccctgtacaatccctttgagtg

SEQ ID NO: 9 - DNA, 18 bp, Artificial Sequence
  Synthetic
tgcttcagtcagctgatg

SEQ ID NO: 10 - DNA, 22 bp, Artificial Sequence
  Synthetic
ttaaattgtcatcttcgtcctt

SEQ ID NO: 11 - DNA, 41 bp, Artificial Sequence
  Synthetic
tcagtactagtgcctgtgcccacaatcgtttttaaacgggt

SEQ ID NO: 12 - DNA, 43 bp, Artificial Sequence
  Synthetic
tcgtatacagggcttttgacatctatcttggaagcgacaacaa

SEQ ID NO: 13 - DNA, 17 bp, Artificial Sequence
  Synthetic
ctgcacttacaccgcaa

SEQ ID NO: 14 - DNA, 22 bp, Artificial Sequence
  Synthetic
gtagctggttttgctaaattcc

SEQ ID NO: 15 - DNA, 17 bp, Artificial Sequence
  Synthetic
ttgatgagctggagcca

SEQ ID NO: 16 - DNA, 18 bp, Artificial Sequence
  Synthetic
caccctcaatgcagagtc

SEQ ID NO: 17 - DNA, 41 bp, Artificial Sequence
  Synthetic
gtgtgaccctgaagactcggttttagccactgactcggatc

SEQ ID NO: 18 - DNA, 45 bp, Artificial Sequence
  Synthetic
cctccgtgatatggctcttcgtttttttcttacatggctctggtc

SEQ ID NO: 19 - DNA, 20 bp, Artificial Sequence
  Synthetic
atgtggatggctgagttgtt

SEQ ID NO: 20 - DNA, 20 bp, Artificial Sequence
  Synthetic
catgctgagtactggacctc

SEQ ID NO: 21 - DNA, 13 bp, Artificial Sequence
  Synthetic
cagccatccacat

SEQ ID NO: 22 - DNA, 30 bp, Artificial Sequence
  Synthetic
gtactgccactaaagcatacaatgtaacac

SEQ ID NO: 23 - DNA, 31 bp, Artificial Sequence
  Synthetic
aatatgcttattcagcaaaatgacttgatct

SEQ ID NO: 24 - DNA, 33 bp, Artificial Sequence
  Synthetic
cagacaaggaactgattacaaacattggccgca

SEQ ID NO: 25 - DNA, 15 bp, Artificial Sequence
  Synthetic
attgcacaatttgcc